(19) United States
(12) Patent Application Publication
(10) Pub. No.: US 2006/0120961 A1
(43) Pub. Date: Jun. 8, 2006

(54) Glycan analysis using deuterated glucose

(75) Inventors: Luke V. Schneider, Half Moon Bay, CA (US); Michael P. Hall, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Target Discovery, Inc., Palo Alto, CA (US)

(21) Appl. No.: 11/262,311

(22) Filed: Oct. 28, 2005

Related U.S. Application Data
(60) Provisional application No. 60/623,521, filed on Oct. 29, 2004.

(51) Int. Cl. A61K 49/00; G01N 33/00

(52) U.S. Cl. 424/9.1; 204/450; 436/86

(57) ABSTRACT

Novel methods and apparatuses are provided for use in identifying glucose metabolic products and determining metabolic flux by administering D₇-glucose to a subject.

### CROSS-REFERENCES TO RELATED APPLICATIONS

This application claims the benefit of U.S. Provisional Patent Application No. 60/623,521, filed Oct. 29, 2004, which is incorporated herein by reference in its entirety for all purposes.

### BACKGROUND OF THE INVENTION

Glycosylation patterns (including sequence and branching structure) are important for many biological reasons. The sialic acid content of glycosylated plasma proteins is a biological indicator for clearance. See Chitlaru, et al., Biochem. J., 336:647-658 (1998); Millar, Atherosclerosis, 154:1-13 (2001). Glycosylation differences of red blood cell surface proteins is critical for triggering the immune response to different blood types. Glycoproteins are also being pursued as drug products (e.g., full-length glycosylated recombinant thrombopoietins). See Haznedaroglu, et al., Clin. Appl. Thromb. Hemost., 8:193-212 (2002). Glycosylation of cell surface proteins have a predominant role in cell-cell and cell-substratum recognition events in multicellular organisms (Jain, Targets, 2:189-90 (2003)), making the understanding of protein glycosylation patterns critically important for diseases like cancer. P-glycoprotein is associated with multiple drug resistance of breast (Zampieri, et al., Anticancer Res.; 22:2253-9 (2002)), and bladder (Nakagawa, et al., J. Urol., 157:1260-4 (1997)) tumors to chemotherapy. Quality control of glycosylation patterns of recombinant drugs is a critical issue because of the potential for pyrophoric reactions and inactivity of the resultant drug product.

Glycosylation also affects protein biomarker discovery. Definitive biomarker validation can lead to better diagnostic or prognostic assays that are generally noninvasive, fast, and inexpensive. However, many biomarker assays are not specific for a given disease or progression of that disease without the aid of additional confirmatory methods, such as invasive biopsy, expensive imaging (MRI), or the requirement of a more time-consuming battery of diagnostic tests. One of the issues that contributes to this lack of specificity and accuracy for any given assay is the fact that glycoforms of a biomarker may exist that are functionally- or clinically-relevant but are not identified by the methodology used to interrogate the biomarker status. For example, it has been recently reported that an altered glycosylation pattern allows the distinction between prostate specific antigen (PSA) from normal and tumor origins. See Peracaula et al., Glycobiology, 13:457-470 (2003). Therefore, a positive ELISA result from a patient that utilizes an antibody against the non-glycosylated portion of the antigen may be falsely indicative of malignancy (i.e., a false positive). Another example comes from the area of breast cancer. CD44 is a multifunctional cell adhesion protein marker that participates in cell-cell and cell-matrix interactions. In one study, 44.2% of breast carcinomas studied strongly reacted with a monoclonal antibody against CD44; however, only one glycosylated variant CD44v3, present in only 21.3% of the carcinomas significantly correlated with the presence of metastases to the lymph nodes. See Rys, et al., Pol. J. Pathol.; 54:243-247 (2003).

Current glycomic methods suitable for both sequence and structure determination are very laborious, time and sample consuming. Current method for unambiguous sequence and structure determination focus upon serial digestion with specific saccharases (Parekh, et al., U.S. Pat. No. 5,667,984), followed by derivatization and HPLC or MS analysis of the digestion products. Structural analysis has been performed by mass spectrometric fragmentation analysis, but no method has yet been reported that can determine all of the linkages and branching patterns of a complex branched oligosaccharide. See Zaia, J., Mass Spectrom. Rev.; 23:161-227 (2004).

The present invention addresses these and other needs.

### BRIEF SUMMARY OF THE INVENTION

It has been discovered that, surprisingly, glucose metabolic products may be identified and metabolic flux may be determined by administering D₇-glucose to a subject using the methods and apparatuses disclosed herein.

In one aspect, the present invention provides a method of identifying a glucose metabolic product. The method includes administering to a subject a D₇-glucose. The D₇-glucose is allowed to be at least partially metabolized by the subject to form a deuterated target metabolite. The deuterated target metabolite is separated from the subject. After separating the deuterated target metabolite from the subject, the deuterated target metabolite is contacted with a mass tag and allowed to attach to the deuterated target metabolite, thereby forming a mass tagged deuterated target metabolite. The mass of the mass tagged deuterated target metabolite is detected thereby providing identification of the glucose metabolic product.

In another aspect, the present invention provides methods and apparatuses for conducting metabolic analyses, including methods for purifying metabolites of interest, screens to identify metabolites that are correlated with certain diseases and diagnostic screens for identifying individuals having, or being susceptible to, a disease.

In some embodiments, the method involves administering a substrate (e.g. a D₇-glucose D₇-glucose/H₇-glucose mixture, or composition including a D₇-glucose/H₇-glucose mixture) to a subject, where the relative ratio of D₇-glucose to H₇-glucose is known prior to administration. The subject is then allowed sufficient time to at least partially metabolize the substrate to form one or more target metabolites. The abundance of the isotope in a plurality of target analytes in a sample taken from the subject is then determined so that a value for the flux of each target analytes can be ascertained. The abundance of the isotope in the target analyte is determined using an analyzer capable of determining the ratio of ¹H to ²H. Examples of such analyzers include mass spectrometers, infrared spectrometers, and nuclear magnetic resonance spectrometers.

### DETAILED DESCRIPTION OF THE INVENTION

Definitions

The term “alkyl,” by itself or as part of another substituent, means, unless otherwise stated, a straight (i.e. unbranched) or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl”.

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen and sulfur atoms may optionally be oxidized and the nitrogen heteroatom may optionally be quaternized. The heteroatom(s) O, N, P and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two heteroatoms may be consecutive, such as, for example, —CH₂—NH—OCH₃ and —CH₂—O—Si(CH₃)₃. As described above, heteroalkyl groups, as used herein, include those groups that are attached to the remainder of the molecule through a heteroatom, such as —C(O)R′, —C(O)NR′, —NR′R″, —OR′, —SR′, and/or —SO2R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (e.g. from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Substituents for the alkyl, heteroalkyl, cycloalkyl, heterocycloalkyl groups can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radicals above, exemplary substituents for the aryl and heteroaryl groups are varied and are selected from, for example: halogen, —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

Two of the substituents on adjacent atoms of the aryl or heteroaryl ring may optionally form a ring of the formula -T-C(O)—(CRR′)_{q}-U-, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ-B-, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), sulfur (S), phosphorus (P), and silicon (Si).

A “glycan” is a molecule having a plurality of monosaccharides joined by glycosidic linkages (e.g. a polysaccharide or oligosaccharide). A “monosaccharide,” as used herein, includes phophosugars in the metabolic pathway.

“Peptide” refers to a polymer in which the monomers are amino acids and are joined together through amide bonds, alternatively referred to as a “polypeptide.” The terms “peptide” and “polypeptide” encompass proteins. Unnatural amino acids, for example, β-alanine, phenylglycine and homoarginine are also included under this definition. Amino acids that are not gene-encoded may also be used in the present invention. Furthermore, amino acids that have been modified to include reactive groups may also be used in the invention. All of the amino acids used in the present invention may be either the D- or L-isomer. The L-isomers are generally preferred. In addition, other peptidomimetics are also useful in the present invention. For a general review, see Spatola, Chemistry and Biochemistry of Amino Acids, Peptides and Proteins, B. Weinstein, eds., Marcel Dekker, New York, p. 267 (1983).

A “substrate,” as used herein, is a composition that includes a D₇-glucose, or a D₇-glucose and H₇-glucose mixture. In some embodiments, the substrate is a D₇-glucose is a composition (e.g. a bolus) having a mixture of D₇-glucose and H₇-glucose. In some embodiments, the ratio of the D₇-glucose and H₇-glucose is known. In some embodiments, the substrate is a D₇-glucose. A D₇-glucose is a glucose having the formula:

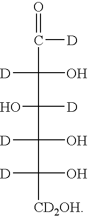

An “H₇-glucose” is a D₇-glucose without deuterium. A “labeled substrate,” as used herein, refers to a composition that includes D₇-glucose. An “unlabeled substrate,” as used herein, refers to a composition that includes H₇-glucose and no D₇-glucose.

A “sample” as used herein, refers to a representative part or a single item derived from a subject. The sample typically contains a deuterated target metabolite. A variety of samples may be analyzed using the present invention. Samples include those materials derived from a bodily, cellular, viral and/or prion source. Some samples are derived from biological fluids such as saliva, blood and urine. In some embodiments, the biological fluids include whole cells, cellular organelles or cellular molecules such as a protein, protein fragment, peptide, carbohydrate or nucleic acid. The biological material can be endogenous or non-endogenous to the source. For example, in one embodiment, the biological material is a recombinant protein harvested from a bacteria and engineered using molecular cloning techniques (see generally, Sambrook et al., Molecular Cloning: A Laboratory Manual, 2d ed. (1989) which is incorporated herein by reference). In another embodiment, the sample comprises a chemically synthesized biological material such as a synthetic protein, protein fragment, peptide, carbohydrate or nucleic acid.

The terms “glycopeptide” and “glycopolypeptide” are used synonymously herein to refer to peptide chains having sugar moieties attached thereto. No distinction is made herein to differentiate small glycopolypeptides or glycopeptides from large glycopolypeptides or glycopeptides. Thus, hormone molecules having very few amino acids in their peptide chain (e.g., often as few as three amino acids) and other much larger peptides and proteins are included in the general terms “glycopolypeptide” and “glycopeptide,” provided they have sugar moieties attached thereto.

The “sequencing” of an oligosaccharide involves deducing certain information concerning the structure of the oligosaccharide such as (i) the type of each monosaccharide unit in the oligosaccharide, (ii) the order in which the monosaccharide units are arranged in the oligosaccharide, (iii) the position of linkages between each of the monosaccharide units (e.g. 1-3, 1-4, etc.), and hence any branching pattern and/or (iv) the orientation of linkage between each of the monosaccharide units (i.e. whether a linkage is an alpha-linkage or a beta-linkage).

The phrase “sequencing a glycan,” refers to the determination of the identification, ordering, and/or location of at least on saccharide unit within a glycan.

A “subject” as used herein generally refers to any living organism from which a sample is taken to conduct an analysis. Subjects include, but are not limited to, microorganisms (e.g., viruses—when in an infected host, bacteria, yeast, molds and fungi), animals (e.g., cows, pigs, horses, sheep, dogs and cats), hominoids (e.g., humans, chimpanzees, and monkeys) and plants. The term includes transgenic and cloned species. The term also includes cell or tissue cultures that can be cultured to carry on the metabolic process under investigation. The term “patient” refers to both human and veterinary subjects.

If the subject is a population of cells or a cell culture, any of the standard cell culture systems known in the art can be used. Examples of suitable cell types include, bust are not limited to, mammalian cells (e.g., CHO, COS, MDCK, HeLa, HepG2 and BaF3 cells), bacterial cells (e.g., E. coli), and insect cells (e.g., Sf9). Further guidance regarding cell cultures is provided in Sambrook et al., Molecular Cloning: A Laboratory Manual, 2d ed. (1989).

A “mass tag” is a chemical entity that does not match the mass of any hexose sugar, polysaccharide produced from hexose sugars, and any deoxy-hexose sugar. In one embodiment the mass tag is a “mass defect tag”, which comprises a tag that incorporates one or more “mass defect atom” or “mass defect element.” A “mass defect atom” or “mass defect element” is defined as an atom having an atomic number from 17 and 77 (inclusive). The mass defect atom/element imparts a mass shift in any tagged glycan that allows such glycans to be discriminated from untagged glycans.

The term “cellular state” includes systemic (tox/efficacy) effects of a drug, chemical, or biochemical exposure as well as disease effect.

Introduction

It has been discovered that, surprisingly, glucose metabolic products may be efficiently and economically identified using a novel combination of stable isotopic tracing and mass tag labeling. The combination of these two methods allows the combined structure and sequence determination of glycoforms. The methods advantageously reduce sample amount requirements and analysis time by eliminating multiple steps relative to the known methods. Both the sequence and the branching structure of a glycoform may be determined simultaneously.

### I. METHODS OF IDENTIFYING A GLUCOSE METABOLIC PRODUCT

In one aspect, the present invention provides a method of identifying a glucose metabolic product. The method includes administering to a subject a D₇-glucose. The D₇-glucose is allowed to be at least partially metabolized by the subject to form a deuterated target metabolite. The deuterated target metabolite is separated from the subject. After separating the deuterated target metabolite from the subject, the deuterated target metabolite is contacted with a mass tag and allowed to attach to the deuterated target metabolite, thereby forming a mass tagged deuterated target metabolite. The mass of the mass tagged deuterated target metabolite is detected thereby providing identification of the glucose metabolic product.

In some embodiments, the mass tagged deuterated metabolite is fragmented using an enzymatic, chemolytic or mass spectrometric fragmentation method to produce a population of labeled mass tagged deuterated metabolite fragments and unlabeled deuterated metabolite fragments. The mass of the labeled mass tagged deuterated metabolite fragments may be detected using any appropriate method, as detailed below. In some embodiments, the labeled mass tagged deuterated metabolite fragments and unlabeled deuterated metabolite fragments are distinguished based on the nuclear binding energy of a mass defect tag.

A. Metabolism of D₇-Glucose

The stable isotope method involves administering D₇-glucose to a subject and allowing the subject to metabolize (or partially metabolize) the D₇-glucose. The D₇-glucose may be enzymatically converted to one or more monosaccharides in vivo (or in situ) as shown, for example, in Scheme 1 below. Each conversion along the metabolic monosaccharide pathway results in the replacement of backbone deuterium ([²H]) with hydrogen ([¹H]). Each replacement of deuterium with hydrogen results in a mass loss of 1.006277 amu.

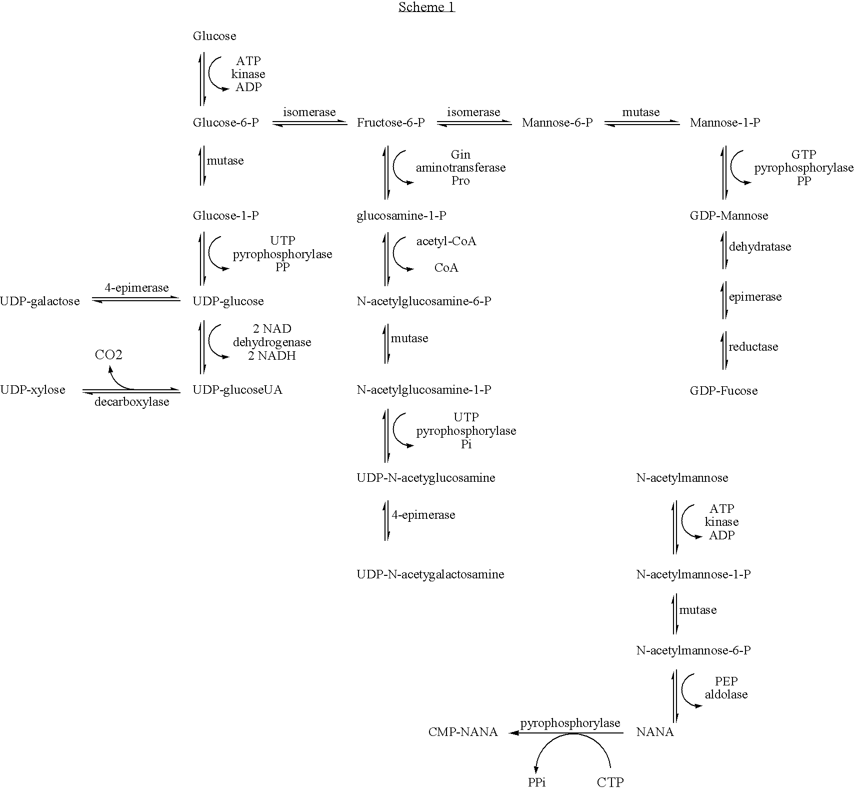

As shown in Table 1 below, the deuterated monosaccharides produced from the metabolism of D₇-glucose will generally differ by at least the mass of one neutron (1.006277 amu). The mass of a single neutron is easily distinguished by mass spectrometry.

Two deuterated monosaccharides produced from the metabolism of D₇-glucose that do not differ by at least 1 amu are mannose and galactose. The mechanism for mannose conversion through fructose involves two isomerizations resulting in a 50:50 chance of deuterium loss at C1. Therefore, two deuterated mannose metabolites are produced from the metabolism D₇-glucose: a lighter isotope with a mass of 185.1 and a heavier isotope with a masse of 186.1 amu. The heavier isotope overlaps the galactose peak at 186.1 amu. The lighter isotope with a mass of 185.1, however, is easily detected (e.g. by mass spectrometry or NMR).

TABLE 1

| | Monoisotopic | | Monoisotopic |
|---|---|---|---|
| [²H]-Saccharide | Mass (Da) | [²H]-Saccharide | Mass (Da) |
| 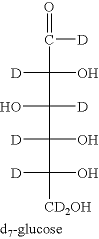 | 187.1 | 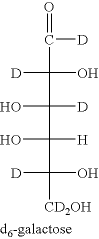 | 186.1 |
| 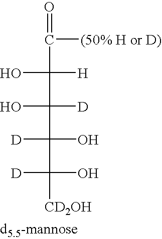 | Doublet: 185.1 186.1 | 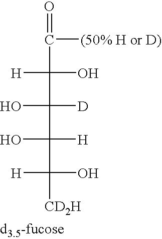 | Doublet: 167.1 168.1 |
| 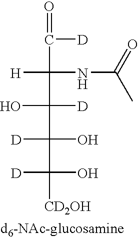 | 226.12 | 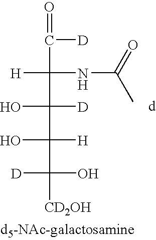 | 227.13 |
| 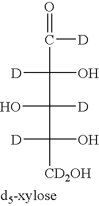 | 155.08 | 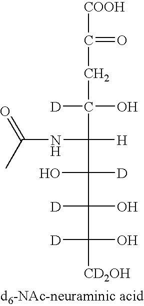 | 313.16 |

A deuterated monosaccharide may be further metabolized by incorporation into a glycan. A glycan may be an independent molecule or attached to another biomolecule, such as a protein or lipid. Typically, the deuterated monosaccharide is circularized and incorporated into the glycan through the action of one or more enzymes, such as a glycosyltransferase. Glycosyltransferases catalyze the addition of activated sugars (donor NDP-sugars), in a step-wise fashion, to a protein, glycopeptide, lipid or glycolipid, or to the non-reducing end of a growing oligosaccharide. A peptide containing a monosaccharide or glycan is a glycopeptide. Glycopeptides may be N-linked or O-linked.

N-linked glycopeptides are peptides having a glycan covalently bound to a peptide asparagine. Typically, the glycan includes a common five sugar structure referred to herein as the trimannosyl core, which is N-linked to asparagine at the sequence Asn-X-Ser/Thr on a peptide chain. N-linked glycopeptides may be formed via a transferase and a lipid-linked oligosaccharide donor Dol-PP-NAG₂Glc₃Man₉ in an en block transfer followed by trimming of the core. In this case the nature of the core saccharide is somewhat different from subsequent attachments. A very large number of glycosyltransferases are known in the art (e.g. a Leloir pathway glycosyltransferase, such as galactosyltransferase, N-acetylglucosaminyltransferase, N-acetylgalactosaminyltransferase, fucosyltransferase, sialyltransferase, mannosyltransferase, xylosyltransferase, glucurononyltransferase and the like).

The trimannosyl core typically includes two N-acetylglucosamine (GlcNAc) residues and three mannose (Man) residues attached to a peptide. Thus, the trimannosyl core typically includes these five sugar residues and no additional sugars, except that it may optionally include a fucose residue. The first GlcNAc is attached to the amide group of the asparagine and the second GlcNAc is attached to the first via a β1,4 linkage. A mannose residue is attached to the second GlcNAc via a β1,4 linkage and two mannose residues are attached to this mannose via an α1,3 and an α1,6 linkage respectively. While the trimannosyl core structure represents an essential feature of N-linked glycans on mammalian peptides, glycan structures on most peptides include other sugars in addition to the trimannosyl core. Methods of the present invention are useful in determining the identity of these and other sugars on a glycopeptide.

O-glycosylation is characterized by the attachment of a variety of monosaccharides in an O-glycosidic linkage to hydroxy amino acids. O-glycosylation is a widespread post-translational modification in the animal and plant kingdoms. The structural complexity of glycans O-linked to proteins vastly exceeds that of N-linked glycans. Serine or threonine residues of a newly translated peptide become modified by virtue of a peptidyl GalNAc transferase in the cis to trans compartments of the Golgi.

The O-linked glycan typically include a core, a backbone region and a peripheral region. The “core” region of an O-linked glycan is the inner most two or three sugars of the glycan chain proximal to the peptide. The backbone region mainly contributes to the length of the glycan chain formed by uniform elongation. The peripheral region exhibits a high degree of structural complexity. The structural complexity of the O-linked glycans begins with the core structure. In most cases, the first sugar residue added at the O-linked glycan consensus site is GalNAc. The sugar may also be GlcNAc, glucose, mannose, galactose or fucose, among others.

In mammalian cells, at least eight different O-linked core structures are found, all based on a core-α-GalNAc residue. O-linked glycans are reviewed, for example, in Montreuil, Structure and Synthesis of Glycopeptides, In Polysaccharides in Medicinal Applications, pp. 273-327, (1996), Eds. Severian Damitriu, Marcel Dekker, NY, and in Schachter and Brockhausen, The Biosynthesis of Branched O-Linked Glycans, 1989, Society for Experimental Biology, pp. 1-26 (Great Britain).

Thus, in some embodiments, the deuterated target metabolite is a deuterated target monosaccharide as described above (e.g. Table 1). The deuterated target monosaccharide may be selected from d₅-xylose, d₆-NAc-neuraminic acid, d₅-NAc-galactosamine acid, d₆-NAc-glucosamine, d₇-glucose, d₆-galactose, d_{3.5}-fucose, and d_{5.5}-mannose (see Table 1 above). In some embodiments, the deuterated target metabolite is a deuterated target monosaccharide hexose sugar, a pentose sugars, or a triose compound resulting from further biochemical conversions of a hexose sugar. Other exemplary target metabolites are discussed, for example in Lee et al., U.S. Patent Application No. 20030180800 and Boros et al., Drug Discovery Today, 7:364-372 (2002), which are herein incorporated by reference in their entirety for all purposes.

In other embodiments, the deuterated target metabolite is a deuterated glycan. The deuterated glycan includes at least one deuterated monosaccharide described above, which may be in circularized or uncircularized form. The deuterated glycan may be an independent molecule. Alternatively, the deuterated glycan may be attached to a protein (to form a deuterated glycopeptide), a lipid (e.g. fat, fatty acid, etc.) to form a deuterated glycolipid), a nucleic acid (e.g. nucleotides, nucleosides, oligonucleotides, etc.), or an organic acid. Therefore, in some related embodiments, the deuterated target metabolite is a deuterated glycopeptide, glycolipid, glycosylated nucleic acid, or glycosylated organic acid. As described below, the sequence, structure, and quantity of a glycan (e.g. mass tagged deuterated target metabolites) may be determined using the methods described herein and the knowledge in the art regarding glucose metabolism.

In some embodiments, the glycan pattern may be identified (also referred to herein as “glycoforms”). Exemplary glycoforms include high mannose types (Man 9), hybrid types (Hy 2), and complex multi-antennary types (Hex 2).

B. Mass Tags

A mass tag (also referred to as a mass label) includes appropriate compounds that may be detected by mass spectrometry, such as mass defect tags (see below). The mass tags may be selected based on their ability to modify the mass of a deuterated target metabolite based on their molecular weight contribution and/or their ionic nature.

Where the deuterated target metabolite includes a deuterated monosaccharide or glycan, the mass tag is typically attached to the reducing end. Thus, mass tags provide an additional means to discriminate the deuterated target metabolite from other molecular species that may be present in a sample. For example, where the deuterated target metabolite is a deuterated target glycan fragment, the mass tag allows the fragments starting from the reducing sugar to be discriminated and the oligosaccharide branching structure to be readily reassembled.

The following properties may be relevant to the selection of a mass tag:
i) a unique mass able to shift the masses to regions of the spectrum with low background;
ii) a fixed positive or negative charge to direct remote charge fragmentation;
iii) robustness under fragmentation conditions;
iv) the efficiently of attachment to the deuterated metabolite under a range of conditions, particularly denaturing conditions, enabling reproducibly and uniformity in tagging; and
v) the ability to increase the ionization efficiency of the deuterated metabolite, or at least does not suppress it.

Exemplary mass tags include substituted or unsubstituted alkyls, substituted or unsubstituted heteroalkyls, substituted or unsubstituted cycloalkyls, substituted or unsubstituted heterocycloalkyls, substituted or unsubstituted aryls, substituted or unsubstituted heteroaryls, nucleic acids, polynucleotides, amino acids, peptides, synthetic polymers, biopolymers, heme groups, dyes, organometallic compounds, steroids, fullerenes, retinoids, carotenoids and polyaromatic hydrocarbons.

Useful synthetic polymeric mass tags include polyethylene glycol, polyvinyl phenol, polypropylene glycol, polymethyl methacrylate, polypropylene, polystyrene, cellulose, sephadex, dextrans, cyclodextrins, polyacrylamides, and derivatives thereof. Synthetic polymers typically contain monomer units including ethylene glycol, vinyl phenol, propylene glycol, methyl methacrylate, and derivatives and combinations thereof.

Biopolymers include those comprising monomer units such as amino acids, non-natural amino acids, peptide mimics, nucleic acids, nucleic acid mimics and analogs, and saccharides and combinations thereof. Thus, where the deuterated target metabolite is a deuterated glycopeptide, the peptide may serve as a mass tag.

In some embodiments, the mass tag includes a stable isotope. The stable isotope may be selected from ¹³C, ²H, ¹⁵N, ¹⁸O, and ³⁴S. The tag may contain a mixture of two or more isotopically distinct species to generate a unique mass spectrometric pattern at each labeled fragment position.

Other exemplary mass tags are discussed, for example in Ness et al. U.S. Pat. No. 6,027,890, Schmidt et al. WO 99/32501, EP 698218B1, U.S. Pat. No. 5,100,778, U.S. Pat. No. 5,667,984, and Aebersold et al. WO 00/11208, each of which are herein incorporated by reference in their entirety for all purposes.

In an exemplary embodiment, the mass tag includes a detection enhancement component. A detection enhancement component refers to a portion of the mass tag that aids in facilitating detection of the deuterated target metabolite. Accordingly, the detection enhancement component can provide a positively charged ionic species under fragmentation conditions in a mass spectrometer ionization chamber, or the component can provide a negatively charged ionic species under fragmentation conditions in a mass spectrometer ionization chamber. For many of the detection enhancement components, the amount of ionized species present will depend on the medium used to solubilize the deuterated metabolite. Preferred detection enhancement components (e.g., species that can generate a positive or negative charge) can be classified into three categories: 1) components that carry “hard” charge, 2) components that carry “soft” charge, and 3) components that provide no charge but are in close proximity to a chemical moiety carrying a “soft” charge.

Components that carry “hard” charge are arrangements of atoms that are substantially ionized under all conditions, regardless of medium pH. “Hard” positively-charged detection enhancement components include, but are not limited to, tetraalkyl or tetraaryl ammonium groups, tetraalkyl or tetraaryl phosphonium groups, and N-alkylated or N-acylated heterocyclyl and heteroaryl (e.g., pyridinium) groups. “Hard” negatively-charged detection components include, but are not limited to, tetraalkyl or tetraacyl borate groups.

Components that carry “soft” charge are arrangements of atoms that are ionized at a pH above or below their pKa, respectively (e.g., bases and acids). Within the context of the current invention, “soft” positive charges include those bases with a pKa of greater than 8, greater than 10, or greater than 12. Within the context of the current invention, “soft” negative charges include those acids with a pKa of less than 4.5, less than 2, or less than 1. At the extremes of pKa, the “soft” charges approach classification as “hard” charges. “Soft” positively-charged detection enhancement components include, but are not limited to, 1°, 2°, and 3° alkyl or aryl ammonium groups, substituted and unsubstituted heterocyclyl and heteroaryl (e.g., pyridinium) groups, alkyl or aryl Schiff base or imine groups, and guanidino groups. “Soft” negatively-charged detection enhancement components include, but are not limited to, alkyl or aryl carboxylate groups, alkyl or aryl sulfonate groups, and alkyl or aryl phosphonate or phosphate groups.

For both “hard” and “soft” charged groups, as will be understood by one of ordinary skill in the art, the groups will be accompanied by counterions of opposite charge. For example, within various embodiments, the counterions for positively-charged groups include oxyanions of lower alkyl organic acids (e.g., acetate), halogenated organic acids (e.g., trifluoroacetate), and organosulfonates (e.g., N-morpholinoethane sulfonate). The counterions for negatively-charged groups include, for example, ammonium cations, alkyl or aryl ammonium cations, and alkyl or aryl sulfonium cations.

Components that are neutral but are in close proximity to chemical moieties that carry “soft” charge (e.g, lysine, histidine, arginine, glutamic acid, or aspartic acid) can also be used as detection enhancement components.

The detection enhancement component of the mass tag can also be multiply charged or capable of becoming multiply charged. For example, a tag with multiple negative charges can incorporate one or singly charged species (e.g. carboxylate) or it can incorporate one or more multiply charged species (e.g., phosphate).

In a similar manner, tags having multiple positive charges can be purchased or prepared using methods accessible to those of skill in the art. For example, a mass tag bearing two positive charges can be rapidly and easily prepared from a diamine (e.g., ethylenediamine). In a representative synthetic route, the diamine is monoprotected using methods known in the art and the non-protected amine moiety is subsequently dialkylated with a species bearing one or more positive charges (e.g., (2-bromoethyl)trimethylammonium bromide (Aldrich)). Deprotection using art-recognized methods provides a reactive labeling species bearing at least two positive charges. Many such simple synthetic routes to multiply charged labeling species will be apparent to one of skill in the art.

In some embodiments, the mass tag is a mass defect tag. A brief description of mass defect tags are presented below. Mass defect tags are discussed in greater detail in copending U.S. Patent Application No. 20020172961, which is assigned to the same assignee as the present application and incorporated by reference in its entirety for all purposes.

C. Mass Defect Tags

A “mass defect tag,” as used herein, refers to a mass tag that is distinguishable based on the nuclear binding energy of the tag. The term “nuclear binding energy” refers to the mass disparity between the calculated and actual nuclear masses of the elements. The nuclear binding energy is defined in the art as the mass equivalent (according to the theory of relativity) of the energy needed to tear a nucleus apart into its constituent isolated nucleons. See Bueche, F., “Principles of Physics” (McGraw-Hill, NY, 1977).

Mass defect tags are particularly useful in distinguishing between the mass of a mass defect tagged deuterated target metabolite and a different molecule having the same number of nucleons as the mass defect tagged deuterated metabolite. A “nucleon” is a proton or neutron in the nucleus of an atom. The molecule having the same number of nucleons as the mass defect tagged deuterated target metabolite may be derived from unlabeled fragmented deuterated target metabolites or any other chemical molecule derived from a sample of the subject, or may simply be a contaminant. Thus, the mass defect tag may be useful in discriminating between chemical noise in a mass spectrum and the mass defect tagged deuterated target metabolite. Where the deuterated glycan target metabolite is a fragment, the method may include distinguishing between the mass of the labeled mass tagged deuterated glycan fragment and a different molecule having the same number of nucleons as the labeled mass tagged deuterated glycan fragment.

The major constituents of target metabolites are: C, H, O, N and S. Mass defect tags typically contain one or more elements incorporated into the tag that contain a nuclear binding energy that substantially differs from those of the elements associated with the target metabolite (e.g., C, H, O, N, P and S). These elements may also be referred to herein as a mass defect element. Although F may be used in some circumstances as a mass defect element (Schmidt et al., WO 99/32501 (1999)), elements having an atomic number from 17 to 77 provide a greater difference in nuclear binding energy and thus broader utility. For example, a single iodine substitution on an aryl group creates a mass defect of 0.1033 amu more than a 5 fold improvement over that of 5 aryl F substitutions. A single I on an aryl ring (C₆H₄I) exhibits a monoisotopic mass of 202.935777 amu. This is 192 ppm different from the nearest combination of stable isotope and heteroatom-containing organic molecule ([¹²C]₉[¹⁵N][¹⁶O]₅) at 202.974687 amu. Therefore, a single substitution of any of the elements that exhibit a mass defect similar to that of I (e.g., atomic numbers between 35 and 63) will yield a discernable mass defect (at the 10 ppm level) to a total mass of 3,891 amu for any combination of organic heteroatoms. Two such elements will exhibit a discernable mass defect to a total mass of 7,782 amu. Three such elements will exhibit a discernable mass defect to a total mass of 11,673 amu. Alternatively, single, double, and triple additions of I (or an equivalent mass defect element) can be discriminated from each other to a total mass of 4,970 amu in a mass spectrum with 10 ppm mass resolution.

Thus, in an exemplary embodiment, the mass defect tag includes a mass defect element selected from elements having an atomic number from 17 to 77. In a related embodiment, the mass defect element selected from elements having an atomic number from 35 and 63. The mass defect element may also be selected from bromine and iodine.

Table 2 provides a non-limiting description of moieties useful as tags of the present invention.

TABLE 2
Generic Mass Defect Label

A moieties carry charge (positive or negative) for MS ionization.
B moieties are mass defect elements.
C moieties are reactive groups for linkage to biomolecules.
A, B, and C moieties are located on a variety of aromatic/aliphatic frameworks.

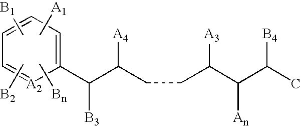

1A. Exemplary Aₙ Moieties:

| | |
|---|---|
| 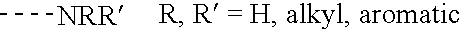 | 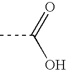 |
| 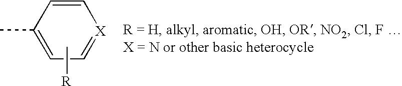 | 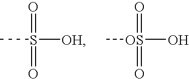 |
| 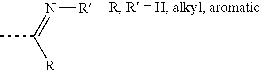 | 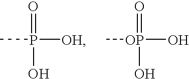 |
| 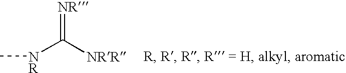 | 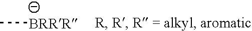 |
| 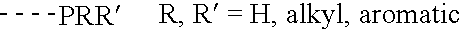 | 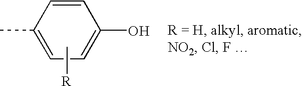 |
| 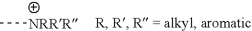 | |
| 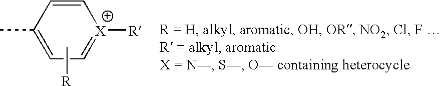 | |
| 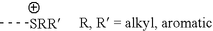 | |
| 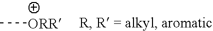 | |

1B. Exemplary Bₙ Moieties:

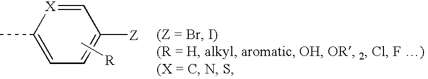

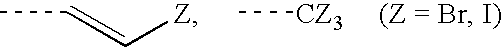

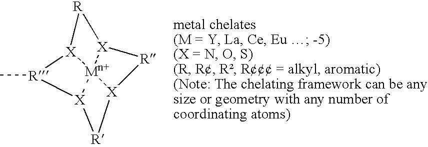

1C. Exemplary C Moieties:

| | |
|---|---|
| 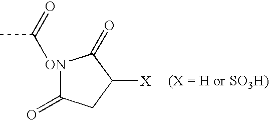 | 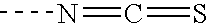 |
| 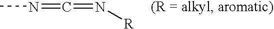 | 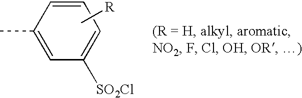 |
| 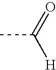 | 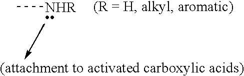 |
| 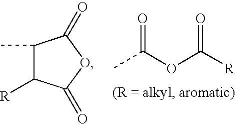 | |

D. Separating the Deuterated Target Metabolite from a Subject

The deuterated target metabolite may be separated from the subject using a wide variety of purification methods know in the art. Typically, a sample containing the deuterated target metabolite is obtained from the subject. The sample may then be subjected to various purification methods known in the art, such as liquid chromatography and gel electrophoresis, to separated the deuterated target metabolite from other components in the sample.

Exemplary purification methods include salt precipitation and solvent precipitation; methods utilizing the difference in molecular weight such as dialysis, ultra-filtration, gel-filtration, and SDS-polyacrylamide gel electrophoresis; methods utilizing a difference in electrical charge such as ion-exchange column chromatography, methods utilizing specific affinity such as affinity chromatography; methods utilizing a difference in hydrophobicity such as reverse-phase high performance liquid chromatography; and methods utilizing a difference in isoelectric point, such as isoelectric focusing electrophoresis. Additional visualization and/or quantification of the isolated or non-isolated proteolytic antibody-phosphonate conjugate may be accomplished using any appropriate technique, including the use of dyes not covalently bound to the phosphonate (e.g. protein dyes such as Commassie Blue). Where gel purification is used, a band containing the deuterated target metabolite can be isolated by excision from the gel using procedures well known to those of skill in molecular biology or biochemistry.

Other useful methods may include the use of ultrafiltration units (e.g. include Amicon or Millipore Pellicon units), solid supports for affinity immobilization or chromatography (e.g. a lectin or antibody molecule bound to a suitable support), anion-exchange resins (e.g. DEAE resins), acrylamide, agarose, dextran, cellulose, cation-exchange resins (e.g. sulfopropyl or carboxymethyl groups), HPLC (e.g. RP-HPLC silica gel having pendant methyl or other aliphatic groups, see e.g. Hardy et al., Proc. Natl. Acad. Sci. USA 85:3289-3293 (1988); Townsend et al., Nature 335:379-380 (1988)), size exclusion chromatography (e.g. Bio-Gel® P-4 gel filtration chromatography, see e.g. Yamashita et al., Meth. Enzymol. 83:105-126 (1982)), capillary gel electrophoresis (see e.g. Gordon et al., Science 242:224-228 (1988)).

E. Separating the Deuterated Glycan from the Target Metabolite

As described above, where the deuterated target metabolite is a glycolipid or glycoprotein, the lipid or protein, respectively, may be used as the mass tag. Alternatively, part or all of the deuterated glycan portion of the glycolipid or glycoprotein target metabolite may be separated from the remainder of the glycolipid or glycoprotein to from a separated deuterated target glycan. The separated deuterated glycan may then be attached to a mass tag at the reducing end, as described below.

A variety of methods may be used to release the deuterated glycan moiety from the target metabolite, including enzymatic (e.g. glycosidases), chemical, and physical techniques (e.g. electron capture dissociation (ECD) and infrared multiphoton dissociation (IRMPD)). These techniques are discussed in more detail below in the context of fragmenting the deuterated target metabolites and are equally applicable to separating the deuterated glycan (or a portion thereof) from the target metabolite.

F. Attaching the Mass Tag to a Deuterated Target Metabolite

The mass tag (including mass defect tags) may be attached to the deuterated target metabolite using any appropriate technique. Where the deuterated target metabolite is a deuterated target monosaccharide, deuterated target glycan, or separated deuterated target glycan, the mass tag is typically covalently attached to the reducing end of the monosaccharide, or glycan.

The mass tags may be covalently attached to the deuterated target metabolite using a reactive functional groups, which can be located at any appropriate position on the mass tag and or deuterated target metabolite. When the reactive group is attached to an alkyl, or substituted alkyl chain tethered to an aryl nucleus, the reactive group may be located at a terminal position of an alkyl chain. Reactive groups and classes of reactions useful in practicing the present invention are generally those that are well known in the art of bioconjugate chemistry: Currently favored classes of reactions available with reactive known reactive groups are those which proceed under relatively mild conditions. These include, but are not limited to. nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

Useful reactive functional groups include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides or reacted with acyl halides;
(h) amine or sulfhydryl groups, which can be, for example, acylated, alkylated or oxidized;
(i) alkenes, which can undergo, for example, cycloadditions, acylation, Michael addition, etc;
(j) epoxides, which can react with, for example, amines and hydroxyl compounds; and
(k) phosphoramidites and other standard functional groups useful in nucleic acid synthesis.

The reactive functional groups can be chosen such that they do not participate in, or interfere with, the attachment reactions disclosed herein. Alternatively, a reactive functional group can be protected from participating in the crosslinking reaction by the presence of a protecting group. Those of skill in the art will understand how to protect a particular functional group from interfering with a chosen set of reaction conditions. For examples of useful protecting groups, See Greene et al., PROTECTIVE GROUPS IN ORGANIC SYNTHESIS, John Wiley & Sons, New York, 1991.

Linkers may also be employed to attach the mass tags to the deuterated target metabolite. Linkers may include reactive groups at the point of attachment to the mass tag and/or the deuterated target metabolite. Any appropriate linker may be used in the present invention, including substituted or unsubstituted alkylene, substituted or unsubstituted heteroalkylene, substituted or unsubstituted cycoalkylene, substituted or unsubstituted heterocycloalkylene, substituted or unsubstituted arylene, and substituted or unsubstituted heteroarylene. Other useful linkers include those having a polyester backbone (e.g. polyethylene glycol), nucleic acid backbones, amino acid backbones, and derivatives thereof. A wide variety of useful linkers are commercially available (e.g. polyethylene glycol based linkers such as those available from Nektar, Inc. of Huntsville, Ala.). Thus, in some embodiments, bifunctional linkers may be employed.

In an exemplary embodiment, the deuterated monosaccharide, glycan, and/or separated glycan target metabolites are be tagged at the reducing sugars using reductive amination chemistry. See Hermanson, G., Bioconjugate Technique (Academic Press: San Diego, Calif.), 1996, pp. 185-186. Reductive amination has been used to successfully to tag the N-terminus of both proteins and polysaccharides. See Hall, M., J. Mass Spectrom.; 38:809-16 (2003); Schneider, Gen. Eng. News 24:28-30 (2004); U.S. Patent Application No. 20020172961.

Tagging may be conducted prior to selective enzymatic hydrolysis of the oligosaccharide or post enzymatic hydrolysis (see fragmenting methods below). In an exemplary embodiment, an deuterated monosaccharide, glycan, or separated glycan target metabolite is tagged with a mass tag before enzymatic hydrolysis and a different mass tag after enzymatic hydrolysis to differentiate the original terminal reducing sugar.

G. Fragmenting Methods

In some embodiments, the mass tagged deuterated metabolite is fragmented using an enzymatic, chemolytic or mass spectrometric fragmentation method to produce a population of labeled mass tagged deuterated metabolite fragments and unlabeled deuterated metabolite fragments. In a related embodiment, the mass tagged deuterated metabolite is a mass tagged deuterated glycan (including mass tagged deuterated separated glycans). The mass of the labeled mass tagged deuterated glycan fragments may be detected using the any appropriate method, as detailed below. In some embodiments, the labeled mass tagged deuterated glycan fragments and unlabeled deuterated glycan fragments are distinguished based on the nuclear binding energy of a mass defect tag.

Mass spectrometric fragmentation refers to the breaking of one or more covalent bonds within a mass spectrometer, typically within the spectrometer ionization chamber of the mass spectrometer. Mass spectrometric fragmentation conditions are well known in the art, and include collision induced fragmentation methods (CID), electron capture dissociation methods (ECD), and infrared multiphoton dissociation methods (IRMPD). See Hakansson K, J Proteome Res. 2(6):581-8 (2003). In an exemplary embodiment, the fragmentation method is a low-energy CID or IRMPD.

Where the deuterated target metabolite is a glycan (including separated glycans), glycolipid, or glycopeptide, low-energy fragmentation generally occurs at the glycosidic ether bonds, particularly in IRMPD methods. By acylating or alkylating the free hydroxyl groups the number and/or the position of branch points in the structure may be identified. The stable deuterium isotopes within monosaccharide units enable identification of glycan subunits. See Table 1. Furthermore, mass tags may be used to identify the reducing end fragments from internally generated scission fragments and fragments from non-reducing ends of complex oligosaccharide antennae. See Zaia, J., Mass Spectrom. Rev. 23:161-227(2004).

The resulting mass spectrum may include a multitude of peaks resulting from chemical noise, including unlabeled deuterated glycan fragments. Mass defect tags may be applied to serial glycolytic methods to further enhance oligosaccharide sequencing and/or identification methods. See U.S. Patent Application No. 20020172961.

A mass spectrum results from the number of ions (counts) that strike a detector plate within the mass spectrometer. The time at which the ions strike the detector plate determines the mass to charge (m/z) ratio of the ion striking the plate. The detector plate is calibrated with known m/z molecules. Each scanning time bin on the detector plate is then assigned an average m/z value and collects ions with m/z ratios of a defined range which is based on the particular design configuration of the instrument. Generally, the size range covered by each detector bin varies as the square root of the m/z value of the bin. This means that the absolute mass precision decreases with increasing m/z in the mass spectrometer. Noise in a mass spectrometer is typically positive. Therefore, the signal is typically greater than or equal to zero in each bin.

In some embodiments, the large number of counts in a mass spectrum of a fragmented deuterated metabolite may require that the mass spectrum be deconvolved by an algorithm. For example, at highly fragmenting conditions virtually all the peaks in the mass spectrum overlay a nearly 1 amu pattern. Algorithms useful in deconvolving the mass spectrum are presented in detail in U.S. Patent Application No. 20020172961.

A variety of enzymes are useful in fragmenting the deuterated target metabolites of the present invention, including peptidases (where the deuterated target metabolite is a glycopeptide), lipases (where the deuterated target metabolite is a glycolipid), and glycosidases (where the deuterated target metabolite is a glycan or separated glycan). Exemplary fragmenting enzymes include, carboxypeptidases (e.g. carboxypeptidase Y), glycanases (e.g. PNGase F), proteases (e.g. serine proteases), esterases, phosphoesterases, fucasidase, galactosidase, hexosaminidase, mannosidase, sialidase, xylosidase, and the like. In some embodiments, enzymatic fragmentation includes the use of a plurality of fragmenting enzymes.

Chemolytic fragmentation refers to those methods in which a covalent bond of a deuterated target metabolite is disrupted using chemical methods, such as acid or base treatment. Chemolytic fragmentation methods are well known in the art and include, for example, those techniques useful in disrupting the amide bonds of a peptide (e.g. mild acidolyis such a 1% TFA) and techniques useful in disrupting the ether bonds of a glycan (e.g. treatment with base such as tetramethylammonium hydroxide).

H. Identifying the Glucose Metabolic Product

Identifying a glucose metabolic product may be accomplished, for example, by detecting the presence of a deuterated metabolic product, and/or quantifying a deuterated metabolic product. Where the deuterated metabolic product is a deuterated glycan or separated glycan target metabolite, identification of the glucose metabolic produce may include at least partially determining the sequence and/or glycoform (e.g. the branching structure) of the glycan. In some embodiments, the sequence and glycoform (or partial sequence and glycoform) are determined using a single method of the present invention.

Identification may be accomplished using any applicable technique capable of distinguishing between the deuterated monosaccharides disclosed above, deuterated glycans, deuterated glycans, and/or mass tagged derivatives thereof. Typically, mass spectrometry and/or NMR (e.g. proton NMR) is employed.

For example, glycans may be sequenced by the invention through modification of the methods described by Parekh et al., U.S. Pat. No. 5,667,984 and Rademacher et al., U.S. Pat. No. 5,100,778. In these methods a mass tag is attached to a purified polysaccharide sample, which is subsequently divided into aliquots that are subjected to different regimes of enzymatic and/or chemolytic cleavage to produce a series of labeled oligosaccharide fragments derived from the polysaccharide parent. These fragments are introduced into a mass spectrometer and the sequence of sugars contained in the parent polysaccharide are determined from the resulting mass ladder generated in the mass spectrum from the random labeled oligosaccharide fragments. It is recognized that increased throughput may be obtained by processing several different samples simultaneously in parallel through the use of different mass tags attached to each unique purified polysaccharide parent sample. In some embodiments, a mass defect tag is used, which may provide an additional advantage in distinguishing the tagged glycans from the chemical noise.

In an exemplary embodiment, the identity of at least one monosaccharide at the reducing end of the glycan or separated glycan is determined. In another exemplary embodiment, the identity of at least 2 monosaccharides at the reducing end of the glycan is determined.

Quantification may be accomplished using any applicable technique known in the art. In some embodiments, a mass spectrometer is used for quantification of the relative abundances of the same molecule obtained from two or more sources in a mass spectrometer (see, for example, WO 00/11208, EP1042345A1, and EP979305A1, which are herein incorporated by reference in their entirety for all purposes). Using this particular methodology, a mass tag can be attached to a deuterated metabolite that differs from the other tags by the replacement of one element with a stable isotope of that element are added to the molecules from each source. The sources are mixed subsequent to tagging and the relative abundance of molecules or the tags from each source are quantified in the mass spectrum. The different isotopes are used to uniquely differentiate the peaks arising for the same molecule from each source. Modification of this method to incorporate one or more mass defect elements into the label may improve this quantification by allowing discrimination between the tagged deuterated oligosaccharides and any chemical noise in the resulting mass spectrum.

The invention can be used in conjuction with protein sequencing methods, such as inverted mass ladder sequencing (see, PCT publication WO 00/11208) and other MS protein sequencing, quantification, and identification methods, such as are outlined in U.S. Pat. No. 6,027,890, and PCT publications WO 99/32501 and WO 00/11208.

One of skill will recognize that the methods of the present invention may be used to detect a plurality of mass tagged deuterated metabolites. In some embodiments, at least two labeled mass tagged deuterated glycan fragments are detected.

I. Methods of Administration

The method by which the D₇-glucose is administered to the subject can vary but should be administered in such a way that the substrate can be metabolized within a reasonable time frame. The D₇-glucose can be administered in substantially pure form or as part of a composition. Compositions can include pharmaceutically acceptable components including, but not limited to, diluents, emulsifiers, binders, lubricants, colorants, flavors and sweeteners, so long as these components do not interfere with the metabolism of the substrate being administered. Guidance on the incorporation of such optional components is discussed, for example, in The Theory and Practice of Industrial Pharmacy (L. Lachman, et al., Ed.) 1976; and Remington's Pharmaceutical Sciences, Mace Publishing Company, Philadelphia, Pa., 17th ed., (1985); and Langer, Science 249:1527-1533 (1990), each of which is incorporated by reference in its entirety.

In some instances, the D₇-glucose is administered orally in solid form (e.g., solid tablet, capsule, powder, pill granule) or as part of a liquid solution (e.g., emulsion, suspension). When shaping into the form of tablets, as the carrier for the substrate, there can be used excipients such as urea, starch, calcium carbonate, kaolin, crystalline cellulose, and potassium phosphate; binders such as water, ethanol, propanol, simple syrup, glucose solution, starch solution, gelatin solution, carboxymethyl cellulose, hydroxypropyl cellulose, methyl cellulose, and polyvinyl pyrrolidone; disintegrators such as carboxymethyl cellulose sodium, carboxymethyl cellulose calcium, low-substitution degree hydroxypropyl cellulose, dried starch, sodium alginate, agar powder, laminaran powder, sodium bicarbonate, and calcium carbonate; surfactants such as polyoxyethylene sorbitan fatty acid ester, sodium lauryl sulfate, and monoglyceride stearate; disintegration inhibitors such as sucrose, stearin, cacao butter, and hydrogenated oil; absorption accelerators such as quaternary ammonium base, and sodium lauryl sulfate; humectants such as glycerin and starch; absorbents such as starch, lactose, kaolin, bentonite, and colloidal silicic acid; and lubricants such as purified talc, stearate, borax and polyethylene glycol. Furthermore, tablets may include a coating, such as sugar, gelatin coated tablets, enteric, or film coatings, as well as double tablets and multilayer tablets.

When shaping into the form of pills, as the carrier for the substrate, there can be used excipients such as glucose, lactose, starch, cacao butter, hardened vegetable oil, kaolin, and talc; binders such as gum arabic, tragacanth powder, gelatin, and ethanol; and disintegrators such as laminarane and agar.

The D₇-glucose, alone or in combination with other suitable components, can also be made into aerosol formulations (e.g., they can be “nebulized”) to be administered via inhalation. Aerosol formulations can be placed into pressurized acceptable propellants, such as dichlorodifluoromethane, propane, nitrogen.

Suitable formulations for rectal administration include, for example, suppositories, which consist of the packaged active ingredient with a suppository base. Suitable suppository bases include natural or synthetic triglycerides, paraffin hydrocarbons, polyethylene glycol, cacao butter, higher alcohols, esters of higher alcohols, gelatin, and semisynthetic glycerides. In addition, it is also possible to use gelatin rectal capsules that consist of a combination of the substrate with a base, including, for example, liquid triglycerides, polyethylene glycols, and paraffin hydrocarbons.

Formulations of the D₇-glucose suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. The compositions are formulated as sterile, substantially isotonic and in full compliance with all Good Manufacturing Practice (GMP) regulations of the U.S. Food and Drug Administration.

When the D₇-glucose is administered to a population of cells, the cells are typically suspended in a matrix containing the D₇-glucose. The matrix is typically an aqueous solution and can also contain other nutrients. Depending upon the number of cells, the cells can be suspended in standard culture flasks or within the wells of a microtiter plate, for example.

J. Methods of Collecting Samples

As noted above, sample may be collected from any appropriate organisms. For example, samples may be collected from tissues or tissue homogenates, fluids of an organism or cells or cell cultures. Generally, samples are obtained from the body fluid of an organism. Such fluids include, but are not limited to, whole blood, plasma, serum, semen, saliva, urine, sweat, spinal fluid, saliva, gastrointestinal fluids, sweat, cerebral fluid, and lacrimal fluids. In some instances, samples are obtained from fecal material, buccal, skin, tissue biopsy or necropsy and hair. Samples can also be derived from ex vivo cell cultures, including the growth medium, recombinant cells and cell components. In comparative studies to identify potential drug or drug targets (see infra), one sample can be obtained from a diseased subject or cells and another sample a non-diseased subject or from non-diseased cells, for example.

1. Collection Options

Certain methods involve withdrawing a sample of blood from the subject. If whole blood is used, the sample typically is lysed by any of the methods known to those of skill in the art including, for example, freezing/thawing the sample. Urine can be collected by collecting the urine of the subject in a clean container. In some instances, a sample is obtained from the breath of an individual (e.g., when the target metabolite is carbon dioxide). A variety of different devices and methods have been developed to collect breath samples. For example, the breath of a subject can be captured by having the subject inflate an expandable collection bag (e.g., a balloon). The sample can then be transferred to a commercially available storage container for subsequent storage and/or transport (e.g., the VACUTAINER manufactured by Becton-Dickenson Company). Other breath collection devices are described in U.S. Pat. Nos. 5,924,995 and 5,140,993, which are incorporated by reference in their entirety. Tissue samples may be obtained by biopsy.

In the case of cell or tissue cultures, cells are collected by centrifugation or filtration and then lysed according to standard protocols (e.g., sonication, blending, pressurization, freeze thawing and denaturation). Alternatively, cells can be collected and lysed by the addition of trichloroacetic acid (to a final concentration of 5-10% weight to volume), or similar use of other membrane lytic solvents (e.g., chloroform, diethyl ether, toluene, acetone, and ethanol). Such membrane lytic solvents can be used to precipitate macromolecular components and selectively solubilize small molecule metabolites as a precursor to subsequent electrophoretic separation techniques.

The in vivo conversion of D₇-glucose to uniquely identify other sugars by the progressive loss of deuteriums can also be used to track the metabolism of these sugars in the cell. The unique deuterated metabolic products being identified by the presence of “extra” deuterium with can be characterized by H-NMR or mass spectrometric methods. In this embodiment, D₇-glucose can be used to measure metabolic fluxes in a living cell or tissue with such measurements being used to diagnose a disease state, identify metabolic pathways involved in or symptomatic of disease, or used to monitor for efficacy or toxicology effects of a drug product. This can be done using a mixed isotope feed of D₇- and H₇-glucose as described in U.S. Pat. No. 6,764,817, which is incorporated in its entirety by reference. It can also be used in place of [¹³C]-labeled glucose as described by Boros et al., Drug Discovery Today, 7:364-372 (2002) and Cascante et al., Nature Biotechnology 20:243-249 (2002), which are also incorporated in their entirety by reference for all purposes.

### II. METHODS FOR ANALYZING METABOLIC PATHWAYS

In another aspect, the present invention provides methods and apparatuses for conducting metabolic analyses (e.g. metabolic flux), including methods for purifying metabolites of interest, screens to identify metabolites that are correlated with certain diseases and diagnostic screens for identifying individuals having, or being susceptible to, a disease.

In an exemplary embodiment, the method involves administering a substrate (i.e. a D₇ glucose or a D₇-glucose/H₇-glucose mixture) to a subject. In an exemplary embodiment, the amount of D₇ glucose is known prior to administration. In another exemplary embodiment, the relative ratio of D₇-glucose to H₇-glucose is known prior to administration. The subject is then allowed sufficient time to at least partially metabolize the substrate to form one or more target metabolites. The abundance of the isotope in a plurality of target analytes in a sample taken from the subject is then determined so that a value for the flux of each target analytes can be ascertained. The abundance of the isotope in the target analyte is determined using an analyzer capable of determining the ratio of ¹H to ²H. Examples of such analyzers include mass spectrometers, infrared spectrometers and nuclear magnetic resonance spectrometers.

A “target analyte,” as used herein, is a substrate or target metabolite. Thus, a plurality of target analytes includes a plurality of substrates, a plurality of target metabolites, or at least one substrate and at least one target metabolite. In some embodiments, the target analyte is a target metabolite. The target metabolite may be a protein, carbohydrate, nucleic acid, amino acid, nucleotide, nucleoside, fatty acid, organic acid, or fat. In some embodiments, the target analyte is a glycoprotein. In some embodiments, the plurality of target analytes include at least 3 target metabolites. In other embodiments, the plurality of target analytes include at least 5 target metabolites.

Prior to determining the abundance of the isotope in the target analytes and corresponding flux values, the target analytes are typically at least partially separated from other components in the sample. This may be accomplished by performing a plurality of electrophoretic separation methods in series, such that samples from fractions obtained after one method are used in a subsequent electrophoretic method. The actual electrophoretic methods employed can vary, but typically include capillary isoelectric focusing electrophoresis, capillary zone electrophoresis and capillary gel electrophoresis. In some instances, separation and elution conditions of the electrophoretic methods are controlled so that separate fractions for one or more classes of metabolites (e.g., proteins, polysaccharides, carbohydrates, nucleic acids, amino acids, nucleotides, nucleosides, fats, fatty acids, and organic acids) are obtained. This simplifies the analysis because one can simply analyze those fractions containing the class of components to which the target analytes belong.

The invention also provides analytic methods for analyzing metabolic pathways in which samples from a subject have been previously obtained. In such instances, certain methods involve separating at least partially a plurality of target analytes from other components contained in the sample obtained from the subject. The target analytes comprise one or more target metabolites resulting from the metabolism of the substrate by the subject. A flux value for each target analyte is determined from knowledge of the isotopic abundance in the substrate prior to it being administered to the subject and by determining the abundance of the isotope in the target analytes.

Methods for screening metabolites to identify those correlated with various cellular states (e.g., certain diseases) are also included in the invention. Certain screening methods include administering a substrate to a test subject and a control subject, the relative isotopic abundance of the D₇-Glucose and H₇-Glucose in the substrate being known and the test subject having a disease under investigation. The substrate is allowed to be at least partially metabolized by the test subject and control subject to form one or more target metabolites. The conditions under which the administering and allowing steps are performed are controlled so that they are the same for the test and control subject. A sample is obtained from the test and control subject and the relative abundance of the isotope in the target analytes determined to obtain a value for the flux of each target analyte. The flux values for the test and control subject are compared, a difference in the flux value for a target analyte in the test subject and corresponding flux value for the control subject indicating that such analyte is potentially correlated with the disease being studied.

When a sample has been previously acquired, certain screening methods involve analyzing a sample from a test subject having a disease, the sample comprising a substrate labeled with a stable isotope administered to the test subject and/or one or more target metabolites resulting from metabolism of the substrate by the test subject. The relative isotopic abundance of D₇-Glucose and H₇-Glucose in the substrate is known at the time of administration, and the analyzing step includes determining the isotopic abundance of the isotope in a plurality of target analytes in the sample to determine a value for the flux of each target analyte. Flux values for the target analytes in the test subject are compared with flux values for a control subject, a difference in a flux value indicating that such analyte is correlated with the disease.

In another embodiment, the method is used for screening for the presence of a disease. Certain of these methods involve administering to a test subject a substrate, the relative abundance of the D₇-Glucose and H₇-Glucose in the substrate being known. Sufficient time is allowed for the substrate to be at least partially metabolized by the test subject to form one or more target metabolites known to be correlated with the disease. A plurality of electrophoretic methods are performed in series to at least partially separate a plurality of target analytes from other biological components in a sample obtained from the test subject, the target analytes comprising the substrate and/or one or more of the target metabolites. Flux values for the target analytes are determined from the abundance of the isotope in that analyte.

The method is simplified when sample is provided. In such instances, certain method include analyzing a sample from a test subject, the sample comprising a substrate and/or one or more target metabolites resulting from metabolism of the substrate by the test subject, the relative amounts of D₇-Glucose and H₇-Glucose in the substrate being known at the time of administration. The analyzing step itself comprises determining the abundance of the isotope in a plurality of analytes in the sample to determine a value for the flux of each analyte, the plurality of analytes comprising the substrate and/or one or more of the target metabolites. For each target analyte, the determined flux value is compared with a corresponding reference flux value for the same target analytes to assess the test subject's risk of disease. The reference value can be representative of a healthy or diseased state.

Certain methods of the invention provide electrophoretic methods for separating various metabolites using a plurality of electrophoretic methods performed in series. Such separation methods can be utilized to conduct various metabolic analyses. For example, certain analytical methods of the invention involve administering a substrate to a subject. The relative amount of D₇-Glucose and H₇-Glucose in the substrate being known prior to administration. After waiting a period of time to permit the substrate to be utilized, a sample is withdrawn from the subject and used to determine the isotopic composition of multiple target analytes, the target analytes comprising the substrate and/or one or more target metabolites formed from the substrate. Typically, samples are obtained from the subject at different time points and the abundance of the isotope determined for the target analytes in each sample. In this way, the isotopic composition of the substrates can be measured as a function of time to allow a flux value for each of the target analytes to be determined. Various methods can be utilized to determine relative isotopic abundance of the isotope in the target analytes, including nuclear magnetic resonance spectroscopy, infrared spectroscopy and mass spectroscopy.

Unlike certain other methods that focus on the concentration of a particular metabolite, certain methods of the invention are designed to determine flux rather than a single concentration value. This simplifies the methods because flux values can be determined from the relative abundance of the isotope label in the target analytes rather than having to determine absolute concentration values. Furthermore, flux determinations provide insight into certain biological processes that are not observable from simple concentration determinations. For example, while concentration values may appear constant, flux can actually be changing. The concentration of any metabolite is determined by the rates of all reactions involving the formation, conversion, and transport of that metabolite. Therefore, increases in any two specific reactions (fluxes) involving both the formation and removal (conversion or transport) of the metabolite can yield the same apparent concentration of the metabolite. Flux can be altered in response to a number of different stimuli, and thus can serve as sensitive indicator of certain cellular states. For example, flux can be altered in response to factors such as physiological state, exposure to toxins and environmental insults, as well as various disease states such as infection, cancer, inflammation and genetic based defects in metabolism. Thus, flux can be used to detect diverse cellular conditions or states that are not necessarily detectable by other methods.

In some methods of the invention, the samples obtained from the subject are purified prior to determining the isotopic abundance of the isotope in the analytes. The purification procedure is used to at least partially remove other components in the cell from the target analytes of interest. Typically, this is accomplished by separating components within the sample by multiple electrophoretic methods (i.e., multiple dimensions) performed in series.

Certain methods combine the electrophoretic separation aspects of the invention with certain mass spectroscopy techniques of the invention. Such arrangements enable relatively complex samples to be sufficiently reduced in complexity so that samples containing a relatively limited number of target analytes can be directly injected into the mass spectrometer to determine the isotopic abundance in the various target analytes of interest. Such systems can be automated to permit high throughput analysis of metabolic samples.

The flux values determined for the various target analytes can be used in a variety of different applications. For example, flux values for various subjects or various physiological conditions (e.g., diseased or normal) can be used directly as inputs into a database. The flux values can also be employed in various screening applications. For example, the flux values from a test subject can be compared with corresponding flux values for a diseased subject to identify potential markers for the disease (e.g., metabolites that appear to be correlated with the disease). Flux values can also be used as an indication of “systemic” effects of drugs (both toxicity and efficacy), chemicals or biochemicals to which a subject has been exposed. Groups of flux values can be used to develop a “fingerprint” for different cellular states. Once a correlation between a disease state and one or more metabolites have been made, flux values for test subjects can be compared with flux values for individuals having different diseases. Lack of a statistically significant difference between the test and diseased subjects indicates that the test subject has the disease or is susceptible to the disease. Changes in metabolic flux can be manifested as a change in the relative amounts of alternative analytes produced from a single substrate at metabolic branch points, and as the rates at which analytes resulting from serial conversions of a single substrate are produced.

A. General

By feeding a tissue, population of cells or an organism with a substrate and following the ratio of isotopic to nonisotopic metabolites in the cell over time, one can generate a quantitative picture of cellular metabolism. The relative metabolic flux can be ascertained by determining the ratio of the amount of isotopically enriched analytes to normal analytes at any given time using a variety of different detectors capable of detecting the relative abundance of different isotopes (e.g., mass spectrometry). At each metabolic branch point, the relative ratio of isotopic to nonisotopic products on each side of the branch point provides an indication of the flux of metabolite diverted into each branch of the metabolic pathway. Following the rate of change of the isotopic ratio in identifiable metabolites along a linear metabolic pathway in pulse labeled cultures provides an estimate of the metabolic flux through each step of the pathway. Metabolites become isotopically enriched in front of slow kinetic steps and remain isotopically poor immediately after these steps. Once specific changes in cellular metabolism, such as induced by toxic challenge or infection, are identified using the techniques described herein, one can synthesize isotopically enriched compounds that can be used as specific diagnostic markers of these metabolic changes, wherein the substrate is only metabolized or fails to be metabolized in response to a specific disease state (see e.g., U.S. Pat. Nos. 4,830,010; 5,542,419; 6,010,846 and 5,924,995, which are herein incorporated by reference in their entirety for all purposes).

Subjects, methods, and modes of administration are discussed in detail above and are equally applicable to the methods of analyzing metabolic pathways. The mixture may contain between 5-95% relative abundance of D₇-Glucose. In an exemplary embodiment, the mixture contains between 25-75% D₇-Glucose. In another exemplary embodiment, the mixture contains about equimolar ratios of D₇-Glucose and H₇-Glucose. In certain methods, the substrate is administered to the subject as a pulse. Pulsed additions or pulsed labeling refers to the timed addition of an isotopically labeled substrate, wherein the relative isotopic abundance of the isotopes is known. Long pulses can be used to estimate net synthesis rates of particular biomolecules starting from the time of the pulse. In this instance, previous biomass contains no label, but new biomass begins to accumulate the isotope in proportion to the abundance of the H₇-Glucose in the substrate. If the pulse duration is long compared to the turnover of the substrate and target analytes of interest, the net synthesis rate is measured. Short pulses (significantly shorter than the turnover rate) may not account for degradation and recycle, thereby providing an estimate of the unidirectional synthesis rate.

Methods of collecting samples are also discussed above and are equally applicable to collecting the target analytes in the methods of analyzing metabolic pathways.

B. Target Analyte Separation

Although the separation methods below are described in reference to the separation of target analytes in the method for analyzing metabolic pathways, they are equally applicable to separations performed in the methods of identifying a glucose metabolic product presented above.

The methods of the invention are amenable to a variety of different electrophoretic methods. The controlled elution techniques whereby defined fractions are separated spatially, physically or by time, and the labeling and detection methods can be utilized in a number of different electrophoretic techniques. As noted below, the number of electrophoretic methods linked in series is typically at least two, but can include multiple additional electrophoretic methods as well. In some instances, each electrophoretic method in the series is different; whereas, in other instances certain electrophoretic methods are repeated at different pH or separation matrix conditions.

Despite the general applicability of the methods, as noted below CIEF, CZE and CGE methods are specific examples of the type of electrophoretic methods that can be utilized according to the methods of the invention. In certain methods, only two methods are performed. Examples of such methods include a method in which CIEF is performed first followed by CGE. Labeling, if performed, is typically performed after CIEF with detection subsequent to elution of components from the CGE capillary. In another system, the first method is CGE and the final method is CZE. Isotope detection generally is not performed until the completion of the final electrophoretic separation. However, as indicated above, UV/VIS or LIF detection may be used during any or all separation dimensions to monitor the progress of the separations, particularly to determine when fractions are to be collected. A third useful approach involves conducting multiple CZE dimensions. These are specific examples of systems that can be utilized; it should be understood that the invention is not limited to these particular systems. Other configurations and systems can be developed using the techniques and approaches described herein.

In a variation of the electrophoresis systems described below, the capillaries are part of or formed within a substrate to form a part of a microfluidic device that can be used to conduct the analyses of the invention on a very small scale and with the need for only minimal quantities of sample. In these methods, physical fractions of samples typically are not collected. Instead, resolved components are separated spatially or by time. Methods for fabricating and moving samples within microfluidic channels or capillaries and a variety of different designs have been discussed including, for example, U.S. Pat. Nos. 5,858,188; 5,935,401; 6,007,690; 5,876,675; 6,001,231; and 5,976,336, all of which are incorporated by reference in their entirety.

1. Preliminary Purification

Depending on the complexity of the sample (e.g. the number and different types of components within the sample), the target analytes (e.g., substrate and/or target metabolites) are first at least partially purified from other components within the sample. If the sample contains cellular debris or other material that might interfere with separation, such materials can be removed using any of a variety of known separation techniques including, for example, forcibly extruding the sample through sieve material, filtration, centrifugation (e.g., density gradient centrifugation), and various chromatographic methods (e.g., gel filtration, ion exchange or affinity chromatography).

Many macromolecules (e.g., proteins and nucleic acids) can be separated from small molecules (e.g., nucleotides, acetyl CoA, mono- and disaccharides, amino acids) by lysing the cells and quantitatively precipitating the macromolecules by treating the lysed cells with cold trichloroacetic acid (e.g., 5-10% TCA weight to volume for 30 min on ice), while most of the small molecules in the cell remain soluble. Additional separation methods are discussed, for example, by Hanson and Phillips (Hanson, R. S. and Phillips, J. A., In: Manual of methods for general bacteriology, Gerhardt et al. (eds.)., Am. Soc. Microbiol., Washington, D.C., p. 328 (1981)).

2. Multidimensional Electrophoresis

Once such initial purification steps have been completed (if necessary), the target analytes are typically further purified by conducting a plurality of electrophoretic methods conducted in series. For optimal performance, samples whose ionic strength is particularly high can be desalted using established techniques such as dialysis and dilution and reconcentration prior to conducting the electrophoretic methods. The methods are said to be conducted in series because the sample(s) electrophoresed in each method are from solutions or fractions containing components electrophoresed in the preceding method, with the exception of the sample electrophoresed in the initial electrophoretic method. Each of the different electrophoretic methods is considered a “dimension”, hence the series constitutes an “multidimensional” separation.

The series of electrophoretic methods are typically conducted in such a way that components in an injected sample for each electrophoretic method of the series are isolated or resolved physically, temporally or spacially to form a plurality of fractions, each of which include only a subset of components contained in the sample. Thus, a fraction refers to a solution containing a component or mixture of components that are resolved physically, temporally or spacially from other components in a sample subjected to electrophoresis. Hence, resolved components can refer to a single component or a mixture of components that are separated from other components during an electrophoretic method. As just noted, samples in the various electrophoretic methods are obtained from such fractions, with the exception of the first electrophoretic method in which the sample is the original sample containing all the components to be separated.

Typically, these multiple electrophoretic methods in the series separate components according to different characteristics. For example, one method can separate components on the basis of isoelectric points (e.g., capillary isoelectric focusing electrophoresis), other methods can separate components on the basis of their intrinsic or induced (through the application of a label to certain ionizable groups) charge-to-mass ratio at any given pH (e.g., capillary zone electrophoresis), whereas other methods separate according to the size of the components (e.g., capillary gel electrophoresis).

Apparatus used to conduct various electrophoretic methods are known in the art and review in detail in U.S. Pat. No. 6,764,817. which is herein incorporated by reference in its entirety for all purposes. The term “capillary” as used in reference to the electrophoretic device in which electrophoresis is carried out in the methods of the invention is used for the sake of convenience. The term should not be construed to limit the particular shape of the cavity or device in which electrophoresis is conducted. In particular, the cavity need not be cylindrical in shape. The term “capillary” as used herein with regard to any electrophoretic method includes other shapes wherein the internal dimensions between at least one set of opposing faces are approximately 2 to 1000 microns, and more typically 25 to 250 microns. An example of a non-tubular arrangement that can be used in certain methods of the invention is the a Hele-Shaw flow cell (see, e.g., U.S. Pat. No. 5,133,844; and Gupta, N. R. et al., J. Colloid Interface Sci. 222:107-116 (2000)). Further, the capillary need not be linear; in some instances, the capillary is wound into a spiral configuration, for example.

Using the methods of the invention, resolved components can be isolated physically, spatially (e.g., spread throughout the electrophoretic medium contained in the separation cavity) and/or temporally (e.g., controlling elution so different components within a sample elute from the capillary at different times). Thus, the methods of the invention can separate mixtures of components as a function of the composition of elution buffers and/or time. The methods are not limited to the spatial separation of components as are certain traditional gel electrophoresis systems (e.g., 2-D gel electrophoresis systems for protein separation or pulsed-field and sequencing gel systems for nucleic acid separations), or two-dimensional thin layer chromatography (2-D TLC) methods (for small molecule metabolite separations). Instead, with controlled elution, fractions can be collected so components within a fraction fall within a range of isoelectric points and electrophoretic mobilities, for example. Controlled elution of components means that methods can be performed in a reproducible fashion. Such reproducibility is important in conducting comparative studies and in diagnostic applications, for example.

During the elution or withdrawing of resolved components, generally only a portion of the electrophoretic medium containing the resolved component is typically collected in any given fraction. This contrasts with certain 2-D methods in which a gel containing all the resolved components (e.g., proteins) is extruded from the separation cavity and the extruded gel containing all the components is used to conduct another electrophoretic separation. This also contrasts with certain 2-D thin layer chromatography methods in which all the metabolites are separated by their relative affinities for the matrix in a line using one solvent system and are reseparated based on altered affinities by a second solvent system applied perpendicularly to the direction of flow of the first solvent system.

Spacially, physically or temporally resolved components obtained at the conclusion of one electrophoretic method are then used as the source of samples for further separation of components contained within the fraction during a subsequent electrophoretic method. As illustrated in FIG. 1, typically samples from different resolved fractions are sequentially electrophoresed on the same capillary. Normally another sample is not applied until the components in the preceding sample are sufficiently withdrawn from the separation cavity so that there is no overlap of components contained in different fractions. Sequential elution of fractions through the same column can significantly reduce or eliminate variations resulting from differences in cross-linking or electric field strength that can be problematic in certain slab gel electrophoretic methods. Hence, sequential separation can further enhance the reproducibility of the methods of the invention. Other methods, however, can be performed in a parallel format, wherein samples from different fractions are electrophoresed on separate capillaries. This approach allows for separations to be completed more quickly. However, the use of multiple capillaries can increase the variability in separation conditions, thereby reducing to some extent reproducibility between different samples.

In certain methods, the electrophoretic methods are conducted so that pools containing similar components are obtained. For example, the electrophoretic conditions can be controlled so that after the first or first few electrophoretic methods at least one pool containing primarily related components is obtained (e.g., a pool containing primarily proteins, polysaccharides, nucleic acids, amino acids, nucleotides, nucleosides, oligosaccharides, phosphorylated mono- or oligosaccharides, fats, fatty acids or organic acids). Pools of related components can be obtained by capitalizing on the distinctive feature of the different classes of components within a cell. For example, some classes of components are primarily singly charged (e.g., phosphorylated mono- or oligosaccharides), whereas others are primarily zwitterionic (e.g., amino acids, proteins, nucleotides and some fats). CIEF can be used to resolve different zwitterionic components and can also be used to separate zwitterionic species from non-zwitterionic species. Large components (e.g., proteins) can be separated from smaller components (e.g., amino acids, mono- and disaccharides, nucleotides and nucleosides) using CGE. Through judicious selection of pH and buffer conditions, one can control the charge on various components and effect separation of components having different charge-to-mass ratios by CZE. For example, certain buffers can be utilized that selectively complex with certain components to introduce a desired charge to the selected components. An example of such a buffer is a borate buffer that can be used to complex to carbohydrates, thereby imparting a negative charge to the carbohydrates present in the sample. Additional details regarding the electrophoretic methods are set forth infra.

By controlling the electrophoretic conditions to initially separate a complex mixture into pools of different classes of components, one can simplify an analysis considerably. For example, if the metabolite of interest is a carbohydrate, by controlling conditions appropriately so that a pool of carbohydrates is obtained (e.g., using borate buffers), one can ignore fractions containing other classes of compounds. Thus, subsequent electrophoretic separations can simply be conducted with a sample from the pool(s) of interest. Alternatively, if the pool of similar compounds is sufficiently small, individual components of the pool can be completely resolved by mass spectrometric means after the electrophoretic separations. Similarly, once conditions have been established for a particular metabolite, it is not necessary to analyze all fractions obtained from the various electrophoretic methods. The reproducibility of the method enables a sample to be taken only from the few fractions obtained adjacent the fraction(s) previously established to contain the target analytes of interest. Nonetheless, because certain methods can be automated, even during initial screening tests, for example, one can quickly analyze all the final fractions. Even scanning the mass spectrum to identify signals for mass fragments of interest can be automated through the use of computer programs to speed analysis.

3. Capillary Isoelectric Focusing Electrophoresis (CIEF)

Isoelectric focusing may also be used to separate target analytes. Isoelectric focusing is an electrophoretic method in which zwitterionic substances such as proteins, nucleotides, amino acids and some fats are separated on the basis of their isoelectric points (pI). The pI is the pH at which a zwitterionic species such as a protein has no net charge and therefore does not move when subjected to an electric field. In the present invention, zwitterionic species can be separated within a pH gradient generated using ampholytes or other amphoteric substances within an electric field. A cathode is located at the high pH side of the gradient and an anode is located at the low pH side of the gradient.

Zwitterionic species introduced into the gradient focus within the pH gradient according to their isoelectric points and then remain there. The focused components can then be selectively eluted as described below. General methods for conducting CIEF are described, for example, by Kilar, F., “Isoelectric Focusing in Capillaries,” in CRC Handbook on Capillary Electrophoresis: A Practical Approach, CRC Press, Inc., chapter 4, pp. 95-109 (1994); and Schwartz, H., and T. Pritchett, “Separation of Proteins and Peptides by Capillary Electrophoresis: Application to Analytical Biotechnology,” Part No. 266923 (Beckman-Coulter, Fullerton, Calif., 1994); Wehr, T., Rodriquez-Diaz, R., and Zhu, M., “Capillary Electrophoresis of Proteins,” (Marcel Dekker, N.Y., 1999), which are incorporated herein by reference in their entirety.

A more detailed description of multidimensional electrophoresis is presented in U.S. Pat. No. 6,764,817.

4. Capillary Zone Electrophoresis (CZE)

Capillary zone electrophoresis is an electrophoretic method conducted in free solution without a gel matrix and results in the separation of charged components (e.g., proteins, amino acids, fatty acids, fats, sugar phosphates, nucleic acids, nucleotides and nucleosides) based upon their intrinsic charge-to-mass ratios. One advantage to CZE methods is the ability to run with solvent systems that would normally be incompatible with typical water soluble gel matrices. Nonaqueous or water miscible solvent systems can be used to improve the solubility of hydrophobic and membrane bound components that would normally not be resolved by aqueous electrophoretic methods. General methods for conducting the method are described, for example, by McCormick, R. M. “Capillary Zone Electrophoresis of Peptides,” in CRC Handbook of Capillary Electrophoresis: A Practical Approach, CRC Press Inc., chapter 12, pp. 287-323 (1994); Jorgenson, J. W. and Lukacs, K. D., J. High Resolut. Chromatogr. Commun. 4:230 (1981); and Jorgenson, J. W. and Lukacs, K. D., Anal. Chem. 53:1298 (1981), each of which is incorporated by reference in its entirety.

A more detailed description of CIEF is presented in U.S. Pat. No. 6,764,817.

5. Capillary Gel Electrophoresis

Capillary gel electrophoresis refers to separations of proteins, nucleic acids, or other macromolecules accomplished by sieving through a gel matrix, resulting in separation according to size. In one format, proteins are denatured with sodium dodecyl sulfate (SDS) so that the mass-to-charge ratio is determined by this anionic surfactant rather than the intrinsic mass-to-charge ratio of the protein (Cantor, C. R. and Schimmel, P. R., Biophysical Chemistry, W. H. Freeman & Co., NY, (1980)). This means that proteins can be separated solely on the basis of size without charge factoring into the degree of separation. The application of general SDS PAGE electrophoresis methods to capillary electrophoresis (CGE) is described, for example, by Hjerten, S., Chromatogr. Rev., 9:122 (1967).

A more detailed description of capillary gel electrophoresis is presented in U.S. Pat. No. 6,764,817.

C. Detection

Once the target analytes have been at least partially purified from other molecules in the sample, the relative abundance of the isotope in the unmetabolized substrate and/or target analytes is determined. Typically, this involves determining the ratio of ¹H to ²H, although other measures of abundance can also be determined.

The measurement of the concentration of the enriched stable isotope can be made according to a variety of options. One approach is to determine the relative abundance of the isotopic label by mass spectrometry. The target analytes generate distinct signals in the mass spectrum according to the mass to charge ratio of the substrate. The relative signal intensities for the different isotopic forms present enables the relative abundance of the different isotopic forms of each target analyte to be calculated, regardless of the absolute concentration of the analyte in the sample.

Methods for analyzing various biological molecules by mass spectrometry have been established. Mass spectrometry can be used according to known methods to determine the masses of relatively small molecules (e.g., nucleosides, nucleotides, mono and di-saccharides) as well as relatively large molecules. Charged or ionizable analytes can be detected by a variety of mass spectrometric methods. Certain methods include electrospray (ESI) and matrix assisted laser desorption ionization (MALDI) methods coupled with time-of-flight (TOF) ion detection. ESI and MALDI are low energy ionization methods, generally resulting in low fragmentation of most analytes, and are suitable for the ionization of the broadest possible array of target analytes. TOF detection is useful because the accuracy of this technique in determining mass generally allows isotopic resolution to the single atomic mass unit level, even for multiply charged species. However, other mass spectrometric ionization and detection techniques can be usefully employed where the analytes are particularly robust to fragmentation, the isotopic differences between labeled and unlabeled analytes is sufficiently large, and/or the number of charge states sufficiently low, to achieve resolution of the labeled and unlabeled analytes. For a detailed description of mass spectrometry relating to carbohydrate detection, see, e.g., Fox, A. and Black, G. E., “Identification and Detection of Carbohydrate Markers for Bacteria”, ACS Symp. Ser. 541: 107-131 (1994).

An alternative to detection by mass spectrometry is to detect the isotope label using infrared (IR) spectroscopy or nuclear magnetic resonance spectroscopy (NMR). Various target analytes can be detected using this approach, including carbon dioxide, for example. IR and NMR methods for conducting isotopic analyses are discussed, for example, in U.S. Pat. No. 5,317,156; Klein, P. et al., J. Pediatric Gastroenterology and Nutrition 4:9-19 (1985); Klein, P., et al., Analytical Chemistry Symposium Series 11:347-352 (1982); and Japanese Patent Publications No. 61-42219 and 5-142146, all of which are incorporated by reference in their entirety.

In certain methods, target analytes partially or completely purified by the electrophoretic methods are subsequently transported directly to an appropriate detector for analyzing the isotopic composition of the target analytes. In some methods, samples are withdrawn from the individual fractions collected during the final electrophoretic separation and injected directly onto a mass spectrometer to determine relative abundances.

The methods disclosed herein for detection are equally applicable to the methods of identifying a glucose metabolic product discussed above.

D. Flux Determination

In general, the flux of metabolites through each reaction step in any given pathway depends on the relative rates of the forward reaction and reverse reactions. As used herein, flux refers to the rate of change in concentration of a target analyte as a function of time and sample size. The metabolic flux through any single metabolic conversions can be determined from the change in the relative abundance (RAₜ) of isotopically labeled analyte over time (t) according to the equation:

$${Flux}_{analyte} = \frac{ln \{ 1 - \frac{{RA}_{t}}{{RA}_{ss}} \}}{( t ) ( unit of sample )}$$

where RAₛₛ is the relative abundance of the labeled metabolite at long times. Relative abundance (RA) is the relative concentrations of isotopically labeled substrate and/or target metabolite (the target analytes) determined from the ratio of the abundances of isotopic label in the target analytes. In some embodiments, the steady-state relative abundance of the isotope can be considered equal to the known ratio in the initial substrate administered to the subject, such that a only a single sample is needed to determine the metabolic flux. In another embodiment, the steady-state relative abundance of the isotope can be predicted from simultaneous solution of the above equation for two or more relative abundance measurements taken from samples taken at different time points. In another embodiment, the steady-state relative abundance of the isotope can be measured directly from samples taken at long times.

It is apparent to those skilled in the art that an alternative form of the above equation can be used to determine the flux of an analyte from the depletion of isotopically labeled analyte or substrate following a reduction in the relative abundance of isotopically labeled substrate. This alternative form is:

$${Flux}_{analyte} = \frac{ln \{ \frac{{RA}_{t} - {RA}_{ss}}{{RA}_{o} - {RA}_{ss}} \}}{( t ) ( unit of sample )}$$

where RAₒ is the initial relative abundance of the isotopically labeled analyte prior to the administration of substrate to change the relative abundance. In one embodiment, RAₒ is measured directly prior to administration of the new substrate. In another embodiment, RAₒ is assumed to be the same as the relative isotope abundance in the substrate administered prior to the change.

The relative metabolic flux of substrate into any metabolic branch (i) in a network of n branched metabolic pathways is determined from the ratio of relative abundances of isotopically labeled analyte appearing in analytes downstream in each branch (j) of the metabolic pathway at any time (t), possibly at long times (e.g., at the steady-state condition), according to the equation:

$${Flux}_{branch}^{i} = \frac{{RA}_{t}^{i}}{{\sum }_{j = 1}^{n} {RA}_{t}^{j}} [ {Flux}_{substrate} ]$$

To determine flux, typically one or more samples are withdrawn from the subject at different predetermined time points. The samples are then treated, optionally purified, and then analyzed as described above to determine one or more values for the relative concentration of the isotopic label in the target analytes at a sampling time(s) (t). These values can then be utilized in the formula set forth above to determine a flux rate for each of a plurality of target analytes. In some instances, the target analytes used to determine flux are all organic compounds (the analytes do not include carbon dioxide, for example).

It is apparent to those skilled in the art that more accurate flux determinations and standard errors of the estimated fluxes can also be made using statistical curve fitting or parameter fitting methods generally known in the art (e.g., Zar, J. H. Biostatistical Analysis, (Prentice-Hall, Englewood Cliffs, N.J., 1974)) and isotopic ratio data obtained from a plurality of samples taken at different times.

The metabolic flux through a pathway depends on the rate determining step(s) within the pathway. Because these steps are slower than subsequent steps in the pathway, a product of a rate determining step is removed before it can equilibrate with reactant. Further guidance on flux and methods for its determination is provided, for example, by Newsholme, E. A. et al., Biochem. Soc. Symp. 43:183-205 (1978); Newsholme, E. A., et al., Biochem. Soc. Symp. 41:61-110 (1976); and Newsholme, E. A., and Sart., C., Regulation in Metabolism, Chaps. 1 and 3, Wiley-Interscience Press (1973).

E. Utilities

The methods and apparatus for analyzing metabolic pathways can be used to separate and detect a variety of different types of metabolic compounds. Consequently, the methods and apparatus can be used in a variety of metabolic applications. For example, the methods can be used to determine the flux of various metabolites. This capability can be used in biochemical, and especially metabolic, research in determining how the flux of metabolites varies as a function of different cellular states or in response to various external stimuli. The methods have value in clinical research by determining how the flux rates of various metabolites can vary between healthy and diseased states.

More specifically, the invention can be used to develop metomic databases. Such databases can include, for example, a register of various metabolites detected for a particular state or physiological condition of a subject. The database can be cross-referenced with additional information regarding the subject and/or the metabolite. For example, concerning the subject, the database can include information on the genus, species, age, race, sex, environmental exposure conditions, health status, sample collection methodology and type of sample. Flux values can be included for each of the metabolites stored in the database and can be cross indexed with metabolite concentration values, enzyme or transport protein concentration values responsible for the metabolic flux, or gene expression values corresponding to the proteins responsible for the metabolic flux.

Where the fluxes of a plurality of analytes are determined that represent separable components of overall cellular metabolism, a metabolic fingerprint of the subject can be obtained. Analytes from separable components of the overall metabolism are functionally defined as compounds sufficiently separated by a series of enzymatic conversion steps that the isotopic enrichment introduced by any single substrate can not be detected above the natural abundance of the isotope in that analyte, such that a second substrate must be introduced to measure the flux. In general, this functional criteria is satisfied if the target analyte is more than 5 conversion steps removed from the added substrate. In certain methods, a plurality of metabolically separable substrates can be administered simultaneously to a subject and a plurality of metabolically separable target analytes detected from a single sample obtained after a predetermined time from the subject. In a variation of such methods, each of the metabolically separable substrates can be labeled with a different stable isotope. For example D₇-glucose/H₇-glucose, [¹⁵N]-phenylalanine, and [¹³C]-acetate can be administered simultaneously to a subject to determine target analyte fluxes in the glycolysis, amino acid, and fatty acid metabolic pathways.

The invention can be employed in various screening applications. For example, the apparatus and methods of the invention can be used to identify metabolites that are correlated with certain cellular states (e.g., certain diseases). For example, the methods can be utilized to identify metabolites whose concentration or flux varies between healthy and diseased individuals or cells. Enzymes responsible for controlling the concentration and flux of such metabolites are thus identified as potential targets for drug therapy, for instance. In like manner, certain methods can be used to undertake toxicology studies to identify which metabolites, and thus the enzyme(s) controlling their formation, are affected by a toxic challenge.

Screening methods to correlate metabolites and certain cellular states are similar to the general analytical methods set forth supra. For instance, a substrate is administered to a test subject having a disease and at least partially metabolized by the test subject. Generally, one then partially or fully separates the target analytes of interest from other components in the sample under evaluation utilizing the various separation techniques described above. The relative abundance of the isotope in the target analytes is determined using a method capable of detecting the different isotopes to determine a flux value for each of the target analytes in the test subject. These determined values are then compared with the corresponding flux values for a control that serves as a reference for flux values in a non-diseased state.

The control can be a value (e.g., an average or mean value) for a control subject(s) (e.g., someone without the disease) determined under similar conditions. Alternatively, the control can be a range of values previously established to be representative of a non-diseased state. A difference (e.g., a statistically significant difference) between flux values for test and control indicates that the particular metabolite is correlated with the disease. Such a metabolite is a “marker” or potential marker for the disease. The flux values for the control subject can be data obtained previously under like conditions to the test, or the flux values can be determined for a control subject undergoing simultaneous treatment with the test subject under identical conditions.

Of course, similar screening methods can be conducted to develop correlations between certain metabolites and cellular states other than disease states. For example, methods can be conducted to identify metabolites that are correlated with particular developmental stages, states resulting from exposure to certain environmental stimuli and states associated with particular therapeutic treatments.

Multiple metabolites found to have a statistically significant difference in flux values between diseased and control subjects (e.g., markers) can be used to develop a “metabolic flux fingerprint” or simply a “fingerprint” for the disease. Such a fingerprint can subsequently be used to diagnosis the disease (see infra). Typically, such a fingerprint includes at least 2, 3, 4, or 5 metabolites found to be correlated with a disease. In other instances, the fingerprint includes at least 6, 7, 8, 9 or 10 such metabolites, and in still other instances 10, 15, or 20 or more such metabolites.

The results from comparative studies are transferable to a variety of diagnostic applications. For example, the “marker” or “fingerprints” can be used to screen or diagnose subjects to determine if they have, or are susceptible to, a particular disease. The methods track those described supra, except that the substrate labeled with the isotope is administered to a subject suspected to have the disease or susceptible to it (or simply an interested individual seeking to determine if they have, or are susceptible to, the disease). Flux values for the test analyte(s) (e.g., a “metabolic profile” for the test subject) are than compared with reference flux values for individual test analytes (markers) or collections of markers (fingerprints).

The reference values to which the determined values are compared can be representative of either a healthy or diseased state. Furthermore, the reference value can be a particular value or a range of values correlated with either a healthy or diseased state. For example, the reference can be a value (e.g., an average or mean value) for a control subject or subjects either having or not having the disease, the reference value determined under conditions similar to those under which the test subject was tested. Alternatively, the reference can be a range of values drawn from a population of control subjects either having or not having the disease.

If the reference is for a normal or healthy state, a difference (e.g., a statistically significant difference) between flux values for test subject and reference indicates that the test subject has, or is at risk of acquiring, the disease. Alternatively, lack of a difference indicates that the test subject does not have the disease and/or is at not at risk for acquiring the disease. If, however, the reference is representative of a diseased state, then a difference (e.g., a statistically significant difference) between test and reference values indicates that the test subject does not have and/or is not at risk of acquiring the disease. Conversely, lack of a indicates that the test subject either has or is susceptible to acquiring the disease.

Diagnostic screens are not limited to simply detecting disease states. The screens can also be used to detect other types of cellular states such as certain developmental states or toxic states, for example.

When conducting such screening tests, typically the analysis can be simplified. For example, once markers for a disease have been identified, one can establish separation conditions such that the fraction(s) containing the markers or interest is(are) known. Thus, during the screening tests, only the components in those particular fractions need to be evaluated. The reproducibility of the separation and detection aspects of the invention facilitate such analyses.

Such screening methods can be conducted for a variety of different diseases. Diseases that can be evaluated with the methods of the invention include, but are not limited to, various types of cancers, autism, microbial and viral infections, and various digestive disorders.

The methods of the invention have further utility in conducting structure activity studies. For example, the methods can be used to determine the effect that certain chemical agents or combination of agents generally have on metabolism and, more specifically, the effect on the flux of certain metabolites of interest. Such tests can identify agents that are disruptive to metabolism and pinpoint the particular metabolites effected. In other applications, once an agent has been tested initially, the agent or combination of agents can be modified and the analysis repeated to determine what effect, if any, the modifications had on metabolism. Such studies can be useful, for example, in making derivatives of a lead compound identified during initial drug screening trials.

Metabolic engineering studies can also be conducted using the methods of the invention. In such studies, a gene involved in metabolism can be genetically engineered to include certain desired mutations, or the promoter of a gene can be genetically engineered to increase or decrease the relative expression level of the gene. Using the methods described herein, one can determine what effect, if any, the genetically engineered changes have on the metabolism of the test subject.

The following examples are offered to illustrate, but not to limit, the claimed invention.

### III. EXAMPLES

### Example 1

The yeast (ATCC Saccharomyces cerevisiae wild type) were grown in Yeast Nitrogen Base (Sigma-Aldrich) spiked with either D₇-D-glucose (Isotec) or normal D-glucose to a final concentration of 0.1 g/mL. The cultures were incubated at 30° C. to confluence and harvested by centrifugation. Exoglucanase is secreted into the growth media was recovered from the supernatant after trichloroacetic acid precipitation, resuspended, and purified from the other exoglucanase isoforms by established HPLC methods. See Larriba et al., Biomol. Eng. 18:132-42 (2001).

Carboxypeptidase Y was recovered in the cell pellet, which was lysed using the Y-PER™ Yeast Protein Extraction Reagent (Pierce). Carboxypeptidase Y was affinity purified from the Y-PER lysate using the commercially-available mAb attached to an AminoLink column (Pierce) following the manufacturer's protocols.

The glycans from each of these glycopeptides were recovered from the purified model proteins by cleavage with PNGase F. These glycans were further purified by HPLC to produce purified glycans. The mass of the purified glycans were compared between the D-glucose fed culture and the D₇-D-glucose fed culture by ESI-MS to demonstrate the incorporation of the metabolized deuterated hexoses into the glycan. Thus, yeast cells were shown to successfully utilize the perdeuterated glucose for metabolic requirements.

### Example 2

An optimal mass defect tag is formulated for sequencing and characterization of complex oligosaccharides using in-source fragmentation in an ESI-TOF mass spectrometer. The linkage chemistry is designed for efficient attachment to the reducing end of the oligosaccharide (i.e., the free aldehyde). A mass defect label for conjugation to the reducing end of an oligosaccharide is designed with consideration of four general attributes: an element from the periodic table with a significant mass defect, a basic site for protonation for positive-ion mode mass spectral ionization, stability to MS fragmentation (i.e., the label withstands the energy needed to fragment the glycosidic bonds), and an appropriate linking moiety to the reducing end aldehyde.

### Example 2.1

Mass defect tags having an aromatic bromides are advantageous due to a natural 50:50 isotope pair (⁷⁹Br and ⁸¹Br), which provides redundancy in the mass spectrum and improves the ability to resolve the mass defect spectrum because of peak pairing.

TABLE 3
Mass Defect Tags with Aromatic Bromides

| Compound | Reactivity | Already Synthesized |
|---|---|---|
| I | Amine | 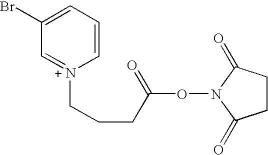 |
| II | | 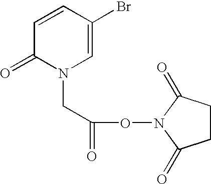 |
| III | Sulfhydryl | 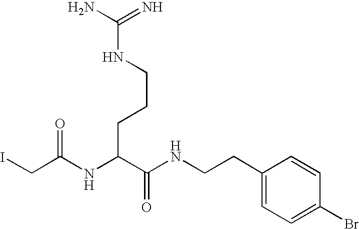 |
| VI | | 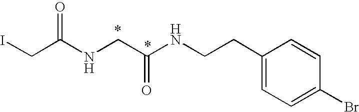 |

A primary amino group is included into the tag for conjugation to the reducing end aldehyde by reductive amination. Incorporation has been demonstrated for numerous UV-absorbent tags such as 2-aminobenzamide and 4-aminobenzoic acid methyl ester into mono- and oligo-saccharides by reductive amination. See Harvey, J. Am. Soc. Mass Spectrom., 11:900-915 (2000). The resulting linkage (a secondary amine) is very stable to mass spectrometric fragmentation conditions, and it provides a basic site for protonation in positive-ion mode ESI mass spectrometry.

Table 4 shows some initial targets for a mass tag. Compound V is commercially-available (Sigma-Aldrich). Compound VI is readily synthesized from commercially-available 5-bromonicotinic acid by lithium aluminum hydride reduction of the corresponding primary amide (FIG. 4). Similarly, other regioisomers are readily synthesizable where the conjugates of this isomer do not possess the desired fragmentation pattern.

Pyridine nitrogen in compound VI is methylated with methyl triflate to form a methyl bromopyridinium species that exhibits good ionization efficiency. Compound IX is methylated to form the trimethylammonium compound with methyliodide or methyl triflate. Compound VII is methylated post-conjugation to the oligosaccharide. The pyridone oxygen has been readily methylated with methyl triflate to give derivatives containing a hard-charged N-methylpyridinium salt.

TABLE 4
Mass Defect Tags

| Compound | Reactivity | Structures |
|---|---|---|
| V | Aldehydes | 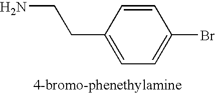 |
| VI | | 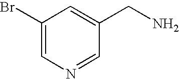 |
| VII | | 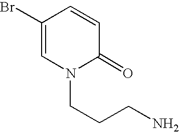 |
| VIII | | 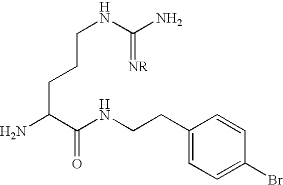 |
| IX | | 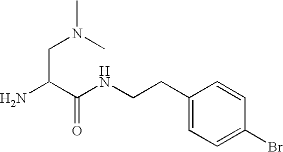 |

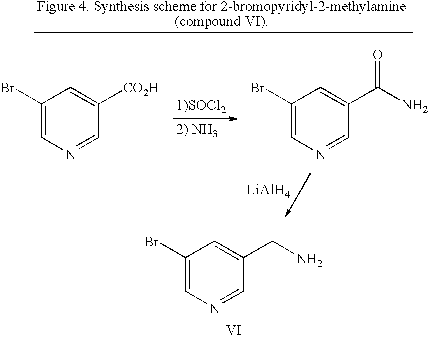

### Example 2.2

The mass tag is conjugated to a commercially-available oligosaccharide. Two purified model branched glycans are commercially-available from Sigma-Aldrich and Ludger: NA2 {mannotriose-di-[N-acetyl-D-glucosamine], bis[galactosyl-(N-acetyl-D-glucosaminyl)]} and A1F {mannotriose-(furcosyl-di-[N-acetyl-D-glucosamine]), mono-sialyl-bis-(galactosyl-N-acetylglucosaminyl)}. The structures are shown below:

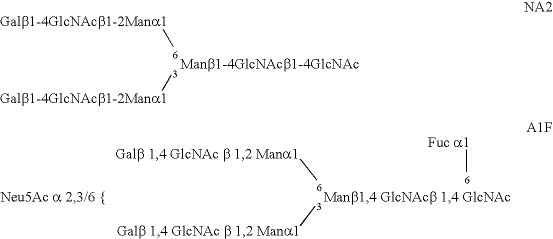

The oligosaccharide is labeled by reductive amination in aqueous solution using the reagents available or synthesized from Table 3. After mass defect labeling of the reducing sugar, the oligosaccharide is permethylated by standard methods. See Dell, Methods Enzymol., 193:647-660 (1990). Permethylation increases ionization of oligosaccharides in mass spectrometry. The modified oligosaccharide is initially dissolved in an appropriate ESI solvent (e.g., 50% (v/v) aqueous acetonitrile containing 1% acetic acid) and subjected to in-source fragmentation in the ionization zone of an ABI Mariner ESI-TOF.

The label is optimized as necessary to: 1) obtain the highest level of oligosaccharide ionization (i.e., lowest limit of detection), 2) ensure that the highest percentage of label remains attached to the fragments and is not cleaved to form a separate label peak.

### Example 3

The purpose of this example is validation of the in vivo d₇-D-glucose stable isotope labeling strategy both for overall effectiveness and specific effectiveness on specific glycans.

### Example 3.1

The cell membrane associated glycans from yeast cultured in d₇-D-glucose are recovered an digested. Yeast membrane proteins are recovered using the Mem-Per®. Eukaryotic Membrane Protein Extraction Reagent Kit (Pierce). The glycans are completely digested by heating overnight in 2M TFA at 100° C. See Gey, et al., Anal, Bioanal, Chem., 356:488-94 (1996). The polymer used for phase separation in the kit forms a separate phase with the lipid soluble fraction after TFA digestion. The resultant monosaccharides are then purified from the aqueous phase by normal-phase HPLC using a Carbohydrate ES column (Alltech) under ambient conditions with a mobile phase of 75% (v/v) aqueous acetonitrile at a flow rate of 1 mL/min and 200 nm detection. The elution time for each monosaccharide is determined with standards. Fractions are collected covering the elution times of each monosaccharide. The monosaccharides are analyzed directly by ESI-TOF by dissolving these fractions in 50:50 acetonitrile:water (with 1% acetic acid). Where sensitivity is problematic, monosaccharide fractions are derivatized with 2-aminobenzamide or a similar species (to generate a basic site for increased ionization) and methylate the hydroxyls to increase the volatility of the oligosaccharide. The ratio of the expected dₓ-D-monosaccharide is compared to the normal D-monosaccharide for each of the monosaccharides in Table 1. Control samples are run from the normal D-glucose culture (processed identically) to verify the peak identities in the mass spectrum.

### Example 3.2

Carboxypeptidase Y (61 kDa) is a high-abundance vaculor glycoprotein required for full protein degradation during sporulation in yeast. Monoclonal antibody specific for carboxypeptidase Y (Molecular Probes (Eugene, Oreg.)), is used for affinity purification of this glycoprotein from yeast lysate. Carboxypeptidase Y has 4 sites of N-glycosylation (residues 13, 87, 168, and 368) (Shimizu, et al., Biosci. Biotechnol. Biochem., 63:1045-50 (1999)), which have been well characterized. See Shimizu, et al., Biosci. Biotechnol. Biochem., 63:1045-50 (1999); Kato, et al., Eur. J. Biochem., 270:4587-93 (2003). Site specific mutants, with alanine substitutions for the native asparagines, have also been generated (Shimizu, et al., Biosci. Biotechnol. Biochem., 63:1045-50 (1999)), thereby simplifying pure glycan generation.

The major yeast exoglucanase (ExgI) carries two short N-linked glycans at residues 165 and 325. See Larriba, et al., FEMS Microbiol. Lett., 15:121-6 (1995). The ExgIb glycoform is the dominant species with minor glycoforms of this exoglucanase arising from underglycosylation of the precursor protein and by elongation of the glycan at Asn₃₂₅. See Larriba, et al., Biomol. Eng., 18:132-42 (2001). Larriba and Cueva have previously proposed this system as an excellent model for glycosylation because the glycoforms are readily separated by HPLC.

Both of the above systems involve N-glycans. N-glycans are easily removed from the purified proteins by the enzyme PNGase F for subsequent analysis. PNGase F is an amidase that cleaves between the innermost GlcNAc and asparagine residues of high-mannose, hybrid, and complex oligosaccharides from N-linked glycoproteins. See Maley, et al., Anal. Biochem., 180:195-204 (1989).

The yeast (either an ATCC Saccharomyces cerevisiae wild type strain or specific carboxypeptidase Y mutant strains from Kyoto University) is grown in Yeast Nitrogen Base (Sigma-Aldrich) spiked with either D₇-D-glucose (Isotec) or normal D-glucose to a final concentration of 0.1 g/mL. The cultures are incubated at 30° C. to confluence and harvested by centrifugation. Exoglucanase is secreted into the growth media and will be recovered from the supernatant after trichloroacetic acid or acetone precipitation, resuspended, and purified from the other exoglucanase isoforms by established HPLC methods. See Larriba, et al., Biomol. Eng., 18:132-42 (2001). Carboxypeptidase Y is recovered in the cell pellet, which will be lysed using the Y-PER™ Yeast Protein Extraction Reagent (Pierce). Carboxypeptidase Y is affinity purified from the Y-PER lysate using the commercially-available mAb attached to an AminoLink column (Pierce) following the manufacturer's protocols.

The glycans are recovered from the purified model proteins by cleavage with PNGase F. Further purification by HPLC is performed as necessary to produce a purified glycan. The structure and sequence of this glycan is determined by conventional serial enzymatic digestion. The production and purification process will be validated with normal D-glucose cultured yeast before we apply the same methods to the D₇-D-glucose cultured sample.

### Example 4

A purified isotope-labeled glycans is attached to a mass defect tag. The resulting tagged glycan is subjected to in-source fragmentation in an ABI Mariner ESI-TOF. The mass of the parent labeled glycan is confirmed before the nozzle potential is increased to attain fragmentation. Oligosaccharide fragmentation begins around 75 V Nozzle potential. The mass defect spectrum is obtained using commercial deconvolution software. The resulting mass defect fragment ions are used to piece together the known sequence. The complete glycan structure and sequence coverage in the mass defect fragmentation spectrum is obtained. Algorithms and software predict unknown glycan sequences and structures from the spectra.

### Example 5

In this example, a high mannose-type oligosaccharide is labeled and sequenced. The oligosaccharide is labeled using methods similar to those described in Parekh, et al., U.S. Pat. No. 5,667,984. Briefly, a mass defect label (2-amino-6-iodo-pyridine) is covalently attached to the reducing terminus of the oligosaccharide in the presence of sodium cyanoborohydride (NaBH₃CN). This incorporates a single mass defect element (iodine) into the parent oligosaccharide. The addition of the mass defect element allows the labeled oligosaccharide fragments to be distinguished from unlabeled fragments and matrix ions in the mass spectrum.

The conjugated oligosaccharide is then aliquoted to reaction tubes containing different saccharases (see Tables 5 and 6) in appropriate reaction buffers. The reactions are allowed to proceed to completion and the resultant reaction products are conjugated at the newly formed reducing ends of the fragments by reaction with the mass defect labels shown for each enzyme (see Table 6), again in the presence of sodium cyanoborohydride. Each of Labels 2 and 3 contain different numbers of mass defect elements, allowing the digest fragments to be distinguished from the terminal fragment of the original oligosaccharide.

TABLE 5
Oligosaccharase Enzymes

| Enzyme # | Species | Enzyme |
|---|---|---|
| 1 | Aspergillus saitoi | α-mannosidase I |
| 2 | Jack bean | α-mannosidase |
| 3 | Achatina saitoi | α-mannosidase II |
| 4 | Jack bean | β-hexosaminidase |
| 5 | Prevotella sp. | β-hexosaminidase |
| 6 | Achatina fulica | β-mannosidase |
| 7 | Streptococcus pneumonae | N-acetyl β-hexosaminidase |
| 8 | Helix pomatia | β-mannosidase |

TABLE 6
Reaction and Label Combinations

| | | Mass Defect |
|---|---|---|
| Enzyme* | Action | Label Used |
| None | None | 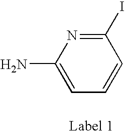 |
| 1 | Cleaves 1 a 2 mannoses at any site | 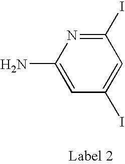 |
| 3 | Cleaves 1 a 3, 6 mannoses to any site Cleaves 1 a 3 mannoses when linked to a branched sugar | 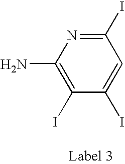 |

*Enzyme number corresponds to the description in Table 5

An aliquot of the Label 3-conjugated reaction mixture (i.e., digested with Enzyme #3) is further digested with Enzyme 1. The reducing sugar termini generated by this reaction are subsequently conjugated to Label 2 as previously described.

Aliquots from all these reactions are then mixed, acidified by the addition of a 50% v/v mixture of 2% acetic acid in methanol and subjected to mass spectral analysis. Because of the low stability of the acetal conjugate in acid solutions mass spectral analysis is conducted immediately after acidification. Alternatively, a different label series that incorporates a hard charge (e.g., an N-alkyl-iodo-pyridinium series) is subjected to mass spectral analysis without acidification. The resulting mass spectrum is deconvolved to remove all chemical noise that does not contain a mass defect labeled peak by the methods of this invention. The resulting deconvolved mass defect spectrum is then algorithmically searched by the methods of this invention by predicting all the possible oligosaccharide sequences that could be attached to each mass defect label used.

The search algorithm calculates the mass for every branch combination of hexose (Hex) and N-acetylaminohexose (HexNAC). Each Hex monomer unit adds a monoisotopic mass unit of 179.055565 amu to the weight of the estimated fragment mass. Each HexNAC monomer unit adds a monoisotopic mass of 220.082114 amu to the estimated fragment mass. There is a net loss of (n−1) times 17.00274 amu for each sugar (n) contained in the fragment. The number of hexoses and N-acetylaminohexoses corresponding to these peaks are shown in Table 7.

TABLE 7
Number and Type of Hexoses Corresponding
the FIG. 1 (A, B, and C) Peaks

Composition

| Peak | HexNAC | Hex |
|---|---|---|
| A | 2 | 1 |
| B | 2 | 5 |
| C | 2 | 9 |
| D | | 1 |
| E | | 1 |
| F | | 2 |
| G | | 3 |

The mass ladder formed from the fragments conjugated to Label 1 indicate that the outermost sugars are be hexoses. The highest mass fragment conjugated to Label 1 corresponds to the parent oligosaccharide. As a result, the four hexose mass difference between the first Label 1-conjugated fragment and the parent indicates the presence of four α-mannoses since both enzyme 1 and enzyme 3 only cleave α-mannoses. Since peak D is the only label 2 conjugate match in FIG. 8B, four of the outermost sugars from the reducing terminus must be 1α2 linked mannoses and there can be no internal 1α2 mannoses.

The next fragment in the Label 1 mass ladder (Peak A) differs by an additional 4 hexoses from the previous fragment. This must correspond to a sample digested with enzyme 3. The only matching Label 3-conjugated fragments are E (a 1 hexose fragment), F (a 2 hexose fragment) and G (a 3 hexose fragment). Since peaks F and G total 5 hexoses, at least one of these fragments must contain a 1α2 linked mannose. Since enzyme 3 only cleaves 1α3 and 1α6 linkages, therefore, there must be at least two separate 1α3 and/or 1α6 linked mannoses in the structure and these mannoses must be interior to the 4 1α2 linked mannoses. From this information the following partial sequence is derived:

{Man₄-1α2}-{Hex₂, Man₂-1α3, 6}-{HexNAC₂, Hex₁}-r

where r indicates the reducing end of the oligosaccharide.

This process is repeated with different enzymes from Table 2 until the complete sequence is determined. For example, digestion with enzyme 3 followed by enzyme 8 allows the determination that the initial sequence is:

-Man-1β4-{HNAC₂}-r

The full sequence of the reducing end of the oligosaccharide is determined by reaction with enzyme 3 followed by enzyme 7.

What is claimed is:

1. A method of identifying a glucose metabolic product, said method comprising:
(a) administering to a subject a D₇-glucose;
(b) allowing the D₇-glucose to be at least partially metabolized by the subject to form a deuterated target metabolite;
(c) separating said deuterated target metabolite from said subject;
(d) after step (c), contacting said deuterated target metabolite with a mass tag and allowing said mass tag to attach to the deuterated target metabolite thereby forming a mass tagged deuterated target metabolite; and
(e) detecting the mass of said mass tagged deuterated target metabolite thereby identifying said glucose metabolic product.

2. The method of claim 1, wherein said deuterated target metabolite is a deuterated monosaccharide.

3. The method of claim 1, wherein said deuterated target metabolite is a deuterated glycan.

4. The method of claim 3, wherein said mass tag is attached at the reducing end of said deuterated glycan to form a mass tagged deuterated glycan.

5. The method of claim 4, further comprising, after step (d) and before step (e), fragmenting the mass tagged deuterated glycan using an enzymatic, chemolytic or mass spectrometric fragmentation method to produce a population of labeled mass tagged deuterated glycan fragments and unlabeled deuterated glycan fragments.

6. The method of claim 5, wherein said mass tag is a mass defect tag comprising a mass defect element having an atomic number from 17 to 77.

7. The method of claim 6, wherein said mass defect element is selected from bromine and iodine.

8. The method of claim 6, wherein said mass defect tag comprises at least two mass defect elements having an atomic number from 17 to 77.

9. The method of claim 6, further comprising, after step (e), distinguishing between the mass of the labeled mass tagged deuterated glycan fragment and a different molecule having the same number of nucleons as the labeled mass tagged deuterated glycan fragment.

10. The method of claim 9, wherein the identity of at least one monosaccharide at the reducing end of the glycan is determined.

11. The method of claim 9, wherein the identity of at least 2 monosaccharides at the reducing end of the glycan is determined.

12. The method of claim 1, wherein the quantity of said mass tagged deuterated target metabolite is determined.

13. The method of claim 5, wherein at least two labeled mass tagged deuterated glycan fragments are detected.

14. The method of claim 1, wherein said subject is a mammal.

15. The method of claim 14, wherein said subject is a cell.

16. The method of claim 3, wherein said deuterated glycan forms part of a glycoprotein.

17. The method of claim 16, further comprising, before step (e), separating the deuterated glycan or a portion of the deuterated glycan from said glycoprotein.

18. The method of claim 1, wherein said separating comprises collecting a sample comprising the deuterated target metabolite from the subject and subjecting said sample to a liquid chromotagraphic procedure to separate the deuterated target metabolite from a sample component.

19. A method for analyzing metabolic pathways, comprising:
(a) administering to a subject a substrate, wherein the relative isotopic abundance of the isotope in the substrate is known;
(b) allowing the labeled substrate to be at least partially metabolized by the subject to form one or more target metabolites; and
(c) determining the abundance of the isotope in a plurality of target analytes in a sample from the subject to determine a value for the flux of each target analyte, wherein the plurality of target analytes comprise the substrate and/or one or more of the target metabolites.

20. The method of claim 19, wherein the determining comprises at least partially separating the target analytes from other biological components in the sample prior to determining the flux values.

21. The method of claim 20, wherein the separating comprises performing a plurality of capillary electrophoresis methods in series.

22. The method of claim 21, wherein the plurality of capillary electrophoresis methods are selected from the group consisting of capillary zone electrophoresis, capillary isoelectric focusing and capillary gel electrophoresis.

23. The method of claim 22, wherein the plurality of capillary electrophoresis methods are selected from the group consisting of capillary zone electrophoresis and capillary isoelectric focusing.

24. The method of claim 23, wherein the performing of the capillary electrophoresis methods comprises performing a plurality of capillary zone electrophoresis methods.

25. The method of claim 24, wherein the performing of the capillary electrophoresis methods generate separate fractions for at least one class of metabolite, wherein the class of metabolite is selected from the group consisting of proteins, polysaccharides, carbohydrates, nucleic acids, amino acids, nucleotides, nucleosides, fats, fatty acids and organic acids.

26. The method of claim 21, wherein the separating comprises conducting a non-electrophoretic separation technique prior to conducting the plurality of electrophoresis methods to precipitate at least some of the biological components.

27. The method of claim 19, wherein the sample is obtained from a bodily fluid, the bodily fluid selected from the group consisting of blood, urine, cerebral fluid, spinal fluid, sweat, and gastrointestinal fluids.

28. The method of claim 19, wherein the sample is a cell, a tissue sample or fecal material.

29. The method of claim 19, wherein the determining comprises obtaining multiple samples from the subject at different predetermined time points, separating the target analytes from other biological components in each of the samples, and determining the abundance of the isotope in the target analytes contained in each sample, whereby a plurality of values for the abundance of the isotope in each target analyte are obtained, the flux value for each target analyte being determined from the plurality of abundance values determined for it.

30. The method of claim 19, wherein the target analytes are selected from the group of proteins, carbohydrates, nucleic acids, amino acids, nucleotides, nucleosides, fatty acids, organic acids, and fats.

31. The method of claim 30, wherein the target analyte is a glycoprotein.

32. The method of claim 19, wherein the plurality of target analytes comprise the substrate and at least one target metabolite.

33. The method of claim 19, wherein the plurality of target analytes is at least 3 target metabolites.

34. The method of claim 33, wherein the plurality of target analytes is at least 5 target metabolites.

35. The method of claim 19, wherein determination of the abundance of the isotope is performed by mass spectrometry, infrared spectrometry or nuclear magnetic resonance spectrometry.

36. The method of claim 35, wherein determination of the abundance of the isotope is performed by mass spectrometry.

37. The method of claim 19, wherein said determining comprises performing a plurality of capillary electrophoresis methods, wherein the plurality of electrophoresis methods are selected from the group consisting of capillary zone electrophoresis, capillary isoelectric focusing and capillary gel electrophoresis followed by mass spectrometry.

38. A method for analyzing metabolic pathways, comprising:
(a) separating at least partially a plurality of target analytes from biological components contained in a sample obtained from a subject, the target analytes comprising a substrate and/or one or more target metabolites resulting from the metabolism of the substrate by the subject, and wherein the relative isotopic abundance of the isotope in the substrate is known; and
(b) determining the abundance of the isotope in a plurality of the target analytes in the sample to determine a value for the flux of each target analyte.

39. The method of claim 38, wherein the separating comprises performing a plurality of capillary electrophoresis methods in series, the capillary electrophoresis methods selected from the group consisting of capillary zone electrophoresis, capillary isoelectric focusing and capillary gel electrophoresis.

40. The method of claim 39, wherein determination of the abundance of the isotope is performed by mass spectrometry

41. A method for screening for metabolites correlated with a disease or cellular state, comprising:
(a) administering to a test subject and a control subject a substrate, wherein the relative isotopic abundance of the isotope in the substrate is known and the test subject has the disease;
(b) allowing the labeled substrate to be at least partially metabolized by the test subject and control subject to form one or more target metabolites, and wherein the conditions under which the administering and allowing steps are performed are the same for the test and control subject; and
(c) obtaining a sample from the test and control subject;
(d) determining for each sample the relative abundance of the isotope in a plurality of target analytes to determine a value for the flux of each target analyte, wherein the target analytes comprise the substrate and/or one or more of the target metabolites; and
(e) comparing the values for flux for the test and control subjects, a difference in the flux value for a target analyte in the test subject and corresponding flux value for the control subject indicating that such analyte is potentially correlated with the disease.

42. The method of claim 41, wherein the determining step comprises at least partially separating the target analytes from other biological components in the sample prior to determining the flux values, the separating comprising separately performing a plurality of capillary electrophoresis methods in series with the samples from the test and control subjects.

43. The method of claim 42, wherein the determination of the isotopic abundance is performed by mass spectrometry.

44. The method of claim 41, wherein the disease is selected from the group consisting of cancer, autism, microbial infection and digestive disorders.

45. A method for screening for metabolites correlated with a disease, comprising:
(a) analyzing a sample from a test subject having the disease, the sample comprising a substrate administered to the test subject and/or one or more target metabolites resulting from metabolism of the substrate by the test subject, the relative isotopic abundance of the isotope in the substrate known at the time of administration, and wherein the analyzing step comprises determining the isotopic abundance of the isotope in a plurality of analytes in the sample to determine a value for the flux of each analyte, wherein the plurality of analytes comprise the substrate and/or one or more of the target metabolites; and
(b) comparing flux values for the analytes with flux values for the same analytes obtained for a control subject, wherein a difference in a flux value for an analyte indicates that such analyte is correlated with the disease.

46. A method for screening for the presence of a disease, comprising:
(a) administering to a test subject a substrate, wherein the relative abundance of the isotope in the substrate is known;
(b) allowing sufficient time for the labeled substrate to be at least partially metabolized by the test subject to form one or more target metabolites known to be correlated with the disease;
(c) performing a plurality of electrophoretic methods in series to at least partially separate a plurality of target analytes from other biological components in a sample obtained from the test subject, wherein the target analytes comprise the substrate and/or one or more of the target metabolites;
(d) determining a flux value for the target analytes, the flux value for each target analyte being determined from the abundance of the isotope in that analyte; and
(e) comparing determined flux values with corresponding reference flux values for the same target analytes to assess the test subject's risk of disease.

47. The method of claim 46, wherein
(i) if the reference flux values are representative of presence and/or susceptibility to the disease, a statistically significant difference between reference values and test values indicates that the test subject does not have and/or is not susceptible to acquiring the disease; and
(ii) if the reference flux values are representative of absence and/or lack of susceptibility to the disease, a statistically significant difference between reference values and test values indicates that the test subject does have, or is susceptible to acquiring, the disease.

48. The method of claim 47, wherein the plurality of electrophoretic methods are selected from the group consisting of capillary gel electrophoresis, capillary zone electrophoresis and capillary gel electrophoresis.

49. A method for screening for the presence of a disease, comprising:
(a) analyzing a sample from a test subject, the sample comprising a substrate and/or one or more target metabolites resulting from metabolism of the substrate by the test subject, the relative isotopic abundance of the isotope in the substrate known at the time of administration, and wherein the analyzing step comprises determining the abundance of the isotope in a plurality of analytes in the sample to determine a value for the flux of each analyte, wherein the plurality of analytes comprise the substrate and/or one or more of the target metabolites; and
(b) for each target analyte, comparing the determined flux value with a range of flux values for that analyte, wherein the range is known to be correlated with the disease and if a determined flux value for a target analyte falls within the range for that target analyte, it indicates that the test subject has the disease or is susceptible to the disease.

50. A method for analyzing metabolites in an initial sample, comprising
(a) performing a plurality of capillary electrophoresis methods in series, each method comprising electrophoresing a sample containing multiple metabolites, whereby a plurality of resolved metabolites are obtained, and wherein
(i) the sample electrophoresed contains only a subset of the plurality of resolved metabolites from the immediately preceding method in the series, except the first method of the series in which the sample is the initial sample, the metabolites in the initial sample potentially containing one or more target analytes;
(ii) the capillary electrophoresis methods are selected from the group consisting of capillary isoelectric focusing electrophoresis, capillary zone electrophoresis and capillary gel electrophoresis; and
(b) analyzing fractions containing resolved metabolites from the final electrophoretic method to detect the presence of the target analytes.

51. The method of claim 50, wherein the one or more target analytes are labeled with an ²H isotopic label, and the analyzing comprises detecting the abundance of the label in each target analyte present.

52. The method of claim 51, wherein the analyzing is performed by mass spectroscopy, infrared spectroscopy or nuclear magnetic resonance spectroscopy.
